(19) United States
(12) Patent Application Publication
(10) Pub. No.: US 2008/0145886 A1
(43) Pub. Date: Jun. 19, 2008

(54) Assay for Cytochrome P450 Isoforms 3A4 and 3A5

(75) Inventors: Ralph Laufer, Rome (IT); Annalise Di Marco, Rieti (IT)

Correspondence Address: MERCK AND CO., INC, P O BOX 2000, RAHWAY, NJ, 07065-0907, US

(73) Assignee: ISTITUTO DI RICERCHE DI BIOLOGIA MOLECOLARE P ANGE, Pomezia (IT)

(21) Appl. No.: 11/663,579

(22) PCT Filed: Oct. 3, 2005
(86) PCT No.: PCT/EP05/10694

§ 371 Date: Mar. 23, 2007

Related U.S. Application Data
(60) Provisional application No. 60/616,942, filed on Oct. 7, 2004.

(51) Int. Cl. C12Q 1/30

(52) U.S. Cl. 435/27

(57) ABSTRACT

A rapid and sensitive radiometric assay for assessing the activity of cytochrome P450 (CYP) 3A4/5 and the potential of an analyte to inhibit CYP3A4/5 activity or induce CYP3A4/5 expression is described. All the steps of the assay, including incubations, product separation, and radioactivity counting are preferably performed in a multiwell format, which can be automated.

### CROSS-REFERENCE TO RELATED APPLICATIONS

This application claims the benefit of U.S. Provisional Application No. 60/616,942, filed Oct. 7, 2004, the contents of which are incorporated herein by reference in their entirety.

### BACKGROUND OF THE INVENTION

(1) Field of the Invention

The present invention relates to an assay for assessing the activity of CYP3A4/5 and the potential of an analyte to modulate CYP3A4/5 activity, e.g., inhibitor of CYP3A4/5 activity or inducer of CYP3A4/5 expression. The assay determines CYP3A4/5 activity or expression by measuring 6β-hydroxylation of testosterone in reactions comprising CYP3A4/5, microsomes comprising CYP3A4, or hepatocytes using testosterone labeled with tritium in the 6β position as a substrate and a sorbent which preferentially binds non-polar compounds such as testosterone to separate the labeled testosterone from tritiated water formed during hydroxylation of the labeled testosterone at the 6β position by CYP3A4/5. The assay is useful for assessing CYP3A4/5 enzymatic activity and CYP3A4/5 inhibition or induction potential of drug candidates in order to exclude potent CYP inhibitors or inducers from further development.

(2) Description of Related Art

The pharmacokinetic and toxicokinetic properties of pharmaceuticals depend in great part on their biotransformation by drug metabolizing enzymes. The main drug metabolizing system in mammals is cytochrome P450 (CYP), a family of microsomal enzymes present predominantly in the liver. Multiple isoforms of CYP catalyze the oxidation of chemicals of endogenous and exogenous origin, including drugs, steroids, prostanoids, eicosanoids, fatty acids, and environmental toxins (Ioannides, In Cytochromes P450. Metabolic and Toxicological Aspects. CRC Press, Boca Raton. (1996)). If a drug that is metabolized by a particular CYP isozyme is co-administered with an inhibitor of that same enzyme, changes in its pharmacokinetics can occur, which can give rise to adverse effect (Bertz and Granneman, Clin. Pharmacokinet. 32: 210-258 (1997); Lin and Lu, Clin. Pharmacokinet. 35: 361-390 (1998); Thummel and Wilkinson, Ann. Rev. Pharmacol. Toxicol. 38: 389-430 (1998); von Moltke et al., Biochem. Pharmacol. 55: 113-122 (1998)). It is therefore important to be able to predict and to prevent the occurrence of clearance changes due to metabolic inhibition. During the drug discovery process, it is routine practice in the pharmaceutical industry to assess CYP inhibition potential of drug candidates in order to exclude potent inhibitors from further development (Lin and Lu, ibid. (1998); Crespi and Stresser, J. Pharmacol. Toxicol. Methods 44: 325-331 (2000); Bachmann and CGhosh, Curr. Drug Metab. 2: 299-314 (2001); Riley, Curr. Opin. Drug Disc. Dev. 4: 45-54 (2001)).

Many CYPs are also strongly inducible by xenobiotics, up to 50 to 100 fold. In drug therapy, there are two major concerns with respect to CYP induction. First, induction may cause a reduction in therapeutic efficacy by decreasing systemic exposure as a result of increased drug metabolism. Second, induction may create an undesirable imbalance between toxification and detoxification as a result of increased formation of reactive metabolites (Lin and Lu, Clin. Pharmacokinet. 35: 361-390 (1998)).

CYP3A4 is the most abundant CYP in human liver and is involved in the metabolism of about 50% of drugs used in human therapy (Guengerich, Ann. Rev. Pharmacol. Toxicol. 39: 1-17 (1999)). Inhibition of CYP3A4 activity can give rise to clinically significant and potentially life threatening drug interactions (Thummel and Wilkinson, Ann. Rev. Pharmacol. Toxicol. 38: 389-430 (1998)). Therefore it has become important to assess a drug candidate for if CYP3A4 inhibitory potential. CYP3A4 induction is probably the most important cause for the documented induction-based drug-CYP interactions (Whitlock et al., In Cytochrome P450: Structure, Mechanism and Biochemistry (Second edition). Ortiz de Montellano (Ed.). Plenum Press, New York (1995). pp. 367-390). Because a drug candidate may have the potential for inducing undesirable CYP3A4 induction-based interactions, it has become important to assess new drug candidates for their CYP3A4 induction potential.

Several assay methods are currently used for determining the potential of drug candidates to inhibit CYP3A4 activity. Each of these methods presents distinct advantages and disadvantages. The most widely used method is the testosterone 6β-hydroxylation assay, which is performed in human liver microsomes (HLM) and is specific for enzymes of the CYP3A family (CYP3A4/5) (Waxman et al., Arch. Biochem. Biophys. 263: 424-436 (1988); Maenpaa et al., J. Steroid Biochem. Mol. Biol. 44: 61-67 (1993); Wang et al., Drug Metab. Dispos. 25: 502-507 (1997); Yamazaki and Shimada, Arch. Biochem. Biophys. 346: 161-169 (1997)). According to recent surveys conducted by reviewers in the Center for Drug Evaluation and Research of the United States Food and Drug Administration, the testosterone 6β-hydroxylation assay represents the most commonly used assay in support of new drug applications (Yuan et al., Clin. Pharmacol. Ther. 66: 9-15 (1999); Yuan et al., Drug Metab. Dispos. 30: 1311-1319 (2002)). The practical challenge posed by this assay is that it requires HPLC separation of the reaction product from the substrate, followed by UV or mass spectrometric detection. This renders the assay relatively laborious, time-consuming, and not ideally suited for screening the large number of compounds typically required in an industrial drug discovery setting.

Several alternative assays, which are amenable to high throughput screening, have been introduced over the past several years. These assays are based on use of fluorogenic or radiolabelled substrates, eliminating the need to use HPLC separation. Probably the most successful of these is a fluorometric assay using 7-benzyloxy-4-trifluoromethylcoumarin as substrate (Crespi and Stresser, J. Pharmacol. Toxicol. Methods 44: 325-331 (2000)). Because the substrate is not specific for CYP3A4, the fluorometric assay cannot be ran with HLM but requires the use of a recombinant enzyane. Alternative fluorometric assays, which use CYP3A4-specific substrates and can, therefore, be performed with HLM, have been described (Chauret et al., Anal. Biochem. 276: 215-226 (1999); Stresser et al., Drug Metab. Dispos. 30: 845-852 (2002)). Even though these fluorometric assays are rapid, easy to perform, and amenable to high throughput screening and automation, they suffer from a number of limitations. First, the use of recombinant CYP instead of ELM, which contain the full complement of CYP isozymes, may give rise to differences in inhibitory potency because test compounds may be subject to metabolism by more than one isozyme, leading to different rates of substrate depletion or formation of inhibitory metabolites. Even when this concern is eliminated by the use of a CYP3A4-specific substrate, a second issue is that CYP3A4 inhibition is substrate-dependent (Kenworthy et al., Br. J. Clin. Pharmacol. 48: 716-727 (1999); Stresser et al., Drug Metab. Dispos. 28: 1440-1448 (2000); Wang et al., Drug Metab. Dispos. 28: 360-366 (2000)). CYP3A4 is a large and complex enzyme that is thought to bind substrates and inhibitors in multiple modes and binding sites (Kenworthy et al., Drug Metab. Dispos. 29: 1644-1651 (2001); Shou et al., Eur. J. Pharmacol. 394: 199-209 (2000); Ekins et al., Trends Pharmacol. Sci. 24: 161-166 (2003)). As a result, inhibitory interactions observed with a non-classical CYP3A4 probe may not be representative of those observed with other substrates (Cohen et al., Drug Metab. Dispos. 31: 1005-1015 (2003)). Finally, fluorescence interference is frequently observed with certain classes of compounds.

The release of tritium that accompanies hydroxylation of a substrate has been used to measure the activity of cytochrome P-450 enzymes (Thompson and Siiteri, J. Biol. Chem. 249: 5364-5372 (1974); Reed and Ohno, J. Biol. Chem. 251: 1625-1631 (1976); Miwa et al., J. Biol. Chem. 255: 6049-6054 (1980); Miyairi and Fishman, J. Biol. Chem. 260: 320-325 (1985); Osawa and Coon, Anal. Biochem. 164: 355-361 (1987)). Draper and co-workers described a CYP3A assay procedure based on this principle, in which tritiated water generated from CYPA-mediated metabolism of [1,2,6,7-³H]-testosterone was separated from the unreacted substrate by charcoal extraction (Draper et al., Drug Metab. Dispos. 26: 305-312 (1998)).

Although in vivo animal models may provide some useful information on the factors that affect the in vitro/in vivo extrapolation of induction data, significant species differences in the inductive response preclude the use of animal models for the assessment of human CYP3A4 induction for new drug candidates. Several in vitro models have been established to assess the potential of CYP3A4 induction for new drug candidates, including liver slices, immortalized cell lines, and primary hepatocytes (Silva et al., Drug Metab. Disp. 26: 490-496 (1998); Kostrubsky et al., Drug Metab. Disp. 27: 887-894 (1999); Maurel, Adv. Drug Dev. Rev. 22: 105-132 (1996); LeCluyse, Eur. J. Pharma. Sci. 13: 343-368 (2001)). Among these models, primary cultures of human hepatocytes have been used extensively by academic and industrial laboratories for evaluating CYP3A4 induction. It is generally believed that the primary hepatocyte culture is the most predictive in vitro model for assessing CYP3A4 induction.

A common method used to assess CYP3A4 induction in human hepatocytes is to incubate the cells for 24 to 78 hours in the presence or absence of a prototypical inducer, such as rifampicin, or in the presence of a test compound. CYP3A4 activity is then determined using the testosterone 6β-hydroxylation assay, using either intact cells or microsomes prepared from the cells. Induction activity of test compounds is usually expressed relative to that of rifampicin. The quantification of the CYP3A4-generated metabolite 6β-hydroxytestosterone requires HPLC analysis coupled to UV or mass spectrometric detection and is therefore not ideally suited for high throughput screening.

In light of the above, a non-HPLC assay for identifying modulators of CYP3A4 activity that could be adapted to high throughput screening format and which is based on use of a classical CYP3A4 substrate such as testosterone would be particularly desirable. Therefore, there remains a need for an assay for identifying CYP modulators that is based on using testosterone as the substrate, is at least as sensitive and specific as the conventional assays, and is readily adaptable to a high throughput screening format. There is also a need for an assay for assessing CYP3A4 activity in hepatocytes.

### BRIEF SUMMARY OF THE INVENTION

The present invention provides a rapid and sensitive radiometric assay for assessing the activity of cytochrome P-450 (CYP) 3A4/5 and the potential of an analyte to inhibit CYP3A4/5 activity or induce CYP3A4/5 expression. The assay is based on detecting the release of tritium as [³H]-H₂O which occurs upon CYP3A4/5-mediated 6β-hydroxylation of testosterone labeled with tritium in the 6β position in the presence of the analyte wherein an increase in the release of tritium over time in hepatocytes or the decrease in the release of tritium over time in reactions comprising CYP3A4/5 indicates that the analyte is a modulator of CYP3A4/5 activity or expression. The method further enables CYP3A4/5 activity in hepatocyte preparations to be determined. In contrast to conventional testosterone 6β-hydroxylation assays, the assay of the present invention does not require HPLC separation and mass spectrometry. Instead, the tritiated water product is separated from tritiated testosterone in a solid-phase extraction process using a sorbent which preferentially binds non-polar compounds such as testosterone. When the tritiated testosterone is labeled solely in the 6, position, both the fractional conversion rate and the sensitivity of the assay is improved. All the steps of the assay, including incubations, product separation, and radioactivity counting are preferably performed in a multiwell format, which can be automated.

Therefore, in one embodiment, the present invention provides a method for identifying an analyte that inhibits activity of cytochrome P450 3A4 or 3A5 (CYP3A4/5), which comprises providing an aqueous mixture comprising CYP3A4/5, tritium-labeled testosterone labeled with tritium at the 6, position, NADPH, optionally an NADPH regenerating system, and the analyte; incubating the aqueous mixture for a time sufficient for the CYP3A4/5 activity to hydroxylate the tritium-labeled testosterone at the 6β position, which produces tritium-labeled water; optionally removing the CYP3A4/5 from the aqueous mixture; applying the aqueous mixture to a sorbent which preferentially binds non-polar compounds such as testosterone to remove the tritium-labeled testosterone from the aqueous mixture; and, measuring amount of the tritium-labeled water in the aqueous mixture with the tritium-labeled testosterone removed wherein a decrease in the amount of the tritium-labeled water in the presence of the analyte compared to the amount of the tritium-labeled water in the absence of the analyte indicates that the analyte inhibits activity of the CYP3A4/5.

In a further aspect of the above embodiment, the sorbent comprises a water-wettable polymer formed by copolymerizing at least one hydrophilic monomer and at least one lipophilic monomer in a ratio sufficient for the polymer to be water-wettable and effective at retaining organic solutes thereon. In further aspects of the above embodiment, the lipophilic monomer comprises a phenyl, phenylene, ether, or C₂-C₁₈ alkyl group. In a further still aspect, the lipophilic monomer is divinylbenzene. In further aspects of the above embodiments, the hydrophilic monomer comprises a saturated, unsaturated, or aromatic heterocyclic group. In a further still aspect, the hydrophilic monomer is N-vinylpyrrolidone. In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In another aspect of the above embodiment, the sorbent comprises a non-polar group bonded to a silica substrate. In a further still aspect, the sorbent comprises one or more silanes selected from the group consisting phenyl silane, dimethylsilane, trimethylsilane, ethyl silane, butyl silane, hexyl silane, octyl silane, and octadecyl silane. In further still aspects, the silica substrate is selected from the group consisting of silica particles and silica gel.

In further aspects of the above embodiment, the tritium-labeled testosterone is labeled at the 1, 2, 6β, and 7 positions. In a particularly preferred aspect of the above embodiments and aspects, the tritium-labeled testosterone is labeled solely at the 6, position.

In a further still embodiment, the present invention provides a method for identifying an analyte that inhibits activity of cytochrome 3A4 or 3A5 (CYP3A4/5), which comprises providing an aqueous mixture comprising CYP3A4/5, tritium-labeled testosterone labeled with tritium solely at the 6, position, NADPH, optionally an NADPH regenerating system, and the analyte; incubating the aqueous mixture for a time sufficient for the CYP3A4/5 activity to hydroxylate the tritium-labeled testosterone at the 6β position, which produces tritium-labeled water; optionally removing the CYP3A4/5 from the aqueous mixture; applying the aqueous mixture to a water-wettable polymer formed by copolymerizing at least one hydrophilic monomer and at least one lipophilic monomer in a ratio sufficient for the polymer to be water-wettable and effective at retaining organic solutes thereon to remove the tritium-labeled testosterone from the aqueous mixture; and, measuring amount of the tritium in the aqueous mixture with the tritium-labeled testosterone removed wherein a decrease in the amount of the tritium in the presence of the analyte compared to the amount of the tritium-labeled water in the absence of the analyte indicates that the analyte inhibits activity of the CYP3A4/5.

In further aspects of the above embodiment, the lipophilic monomer comprises a phenyl, phenylene, ether, or C₂-C₁₈ alkyl group. In a further still aspect, the lipophilic monomer is divinylbenzene.

In further aspects of the above embodiment, the hydrophilic monomer comprises a saturated, unsaturated, or aromatic heterocyclic group. In a further still aspect, the hydrophilic monomer is N-vinylpyrrolidone.

In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In a further aspect of the above embodiment, the testosterone is labeled solely at the 6β position by incubating in a reaction mixture testosterone-3-ethyleneacetal and peracetic acid; separating 5β,6β-epoxide (5β,6β-epoxy-17β-hydroxyandrostan-3-one,3-ethyleneacetal) from 5α,6α-epoxide (5α,6β-Epoxy-17β-hydroxyandrostan-3-one,3-ethyleneacetal) produced in the reaction mixture; incubating the 5α,6α-epoxide in tetrahedrofuran containing [³H]-lithium aluminum to produce [6β-³H]-3,3-ethylenedioxyandrostane-5α,17β-diol; and incubating the [6β-³H]-3,3-ethylenedioxyandrostane-5α,17β-diol in an aqueous mixture of acetic acid to produce the [6β-³H]-testosterone.

In a further embodiment, the present invention provides a method for identifying an analyte that inhibits activity of cytochrome 3A4 or 3A5 (CYP3A4/5), which comprises providing an aqueous mixture comprising CYP3A4/5, tritium-labeled testosterone labeled with tritium at the 6β position, NADPH, optionally an NADPH regenerating system, and the analyte; incubating the aqueous mixture for a time sufficient for the CYP3A4/5 activity to hydroxylate the tritium-labeled testosterone at the 6β position, which produces tritium-labeled water; optionally removing the CYP3A4/5 from the aqueous mixture; applying the mixture to a water wettable polymer formed by copolymerizing divinylbenzene and N-vinylpyrrolidone at a ratio of divinylbenzene to N-vinylpyrrolidone such that the poly(vinylbenzene-co-N-vinylpyrrolidone formed is water-wettable and effective at retaining organic solutes thereon to remove tritium-labeled testosterone from the aqueous mixture; and measuring amount of the tritium in the aqueous mixture with the tritium-labeled testosterone removed wherein a decrease in the amount of the tritium in the presence of the analyte compared to the amount of the tritium in the absence of the analyte indicates that the analyte inhibits activity of the CYP3A4/5.

In further aspects of the above embodiment, the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In further aspects of the above embodiment, the tritium-labeled testosterone is labeled at the 1, 2, 60, and 7 positions. In a particularly preferred aspect of the above embodiments and aspects, the tritium-labeled testosterone is labeled solely at the 6β position.

In further still embodiments of the above, the sorbent or water wettable polymer is packed inside a solid phase extraction cartridge or column. In a particularly preferred embodiment of any one of the above, the method is performed in a multiwell plate format comprising a first multiwell plate for performing the incubation, a multicolumn plate in the same configuration as the multiwell plate for separating the labeled testosterone from the tritiated water after the incubation, and a second multiwell plate for collecting the column void volume and washes from the multicolumn for determining the tritium therein.

The present invention further provides a method for identifying an analyte that inhibits activity of cytochrome 3A4 or 3A5 (CYP3A4/5), which comprises providing a multiwell plate and a column plate having an array of solid phase extraction cartridges or columns having therein a sorbent which preferentially binds non-polar compounds such as testosterone; applying to each of the wells of the multiwell plate an aqueous mixture comprising CYP3A4/5, tritium-labeled testosterone labeled with tritium at the 6β position, and an analyte; contacting NADPH and optionally an NAPDH regenerating system to the aqueous mixture in each of the wells above and incubating for a time sufficient for the CYP3A4/5 to hydroxylate the tritium-labeled testosterone at the 6, position, which produces tritium labeled water; optionally separating the CYP3A4/5 from the aqueous mixture in each of the wells of the multiwell plate; applying each aqueous mixture to a separate minicolumn of the column plate to remove the tritium-labeled testosterone from the aqueous mixture; and, measuring amount of the tritium in the aqueous mixture with the tritium-labeled testosterone removed wherein a decrease in the amount of the tritium in the presence of the analyte compared to the amount of the tritium in the absence of the analyte indicates that the analyte inhibits activity of the CYP3A4/5.

In a further aspect of the above embodiment, the sorbent comprises a water-wettable polymer formed by copolymerizing at least one hydrophilic monomer and at least one lipophilic monomer in a ratio sufficient for the polymer to be water-wettable and effective at retaining organic solutes thereon. In further aspects of the above embodiment, the lipophilic monomer comprises a phenyl, phenylene, ether, or C₂-C₁₈ alkyl group. In a further still aspect, the lipophilic monomer is divinylbenzene. In further aspects of the above embodiments, the hydrophilic monomer comprises a saturated, unsaturated, or aromatic heterocyclic group. In a further still aspect, the hydrophilic monomer is N-vinylpyrrolidone. In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In another aspect of the above embodiment, the sorbent comprises a non-polar group bonded to a silica substrate. In a further still aspect, the sorbent comprises one or more silanes selected from the group consisting phenyl silane, dimethylsilane, trimethylsilane, ethyl silane, butyl silane, hexyl silane, octyl silane, and octadecyl silane. In further still aspects, the silica substrate is selected from the group consisting of silica particles and silica gel.

In further aspects of the above embodiment, the tritium-labeled testosterone is labeled at the 1, 2, 6β, and 7 positions. In a particularly preferred aspect of the above embodiments and aspects, the tritium-labeled testosterone is labeled solely at the 6, position.

In a further embodiment, the present invention provides a method for identifying an analyte that inhibits activity of cytochrome 3A4 or 3A5 (CYP3A4/5), which comprises providing a multiwell plate and a column plate having an array of solid phase extraction cartridges or columns having therein a water wettable polymer formed by copolymerizing divinylbenzene and N-vinylpyrrolidone at a ratio of divinylbenzene to N-vinylpyrrolidone such that the poly(vinylbenzene-co-N-vinylpyrrolidone formed is water-wettable and effective at retaining organic solutes thereon; applying to each of the wells of the multiwell plate an aqueous mixture comprising CYP3A4/5, tritium-labeled testosterone labeled with tritium at the 6, position, and an analyte; contacting NADPH and optionally an NAPDH regenerating system to the aqueous mixture in each of the wells and incubating for a time sufficient for the CYP3A4/5 to hydroxylate the tritium-labeled testosterone at the 6β position, which produces tritium-labeled water; optionally separating the CYP3A4/5 from the aqueous mixture in each of the wells of the multiwell plate; applying each aqueous mixture to a separate minicolumn of the column plate to remove the tritium-labeled testosterone from the aqueous mixture; and, measuring amount of the tritium in the aqueous mixture with the tritium-labeled testosterone removed wherein a decrease in the amount of the tritium in the presence of the analyte compared to the amount of the tritium in the absence of the analyte indicates that the analyte inhibits activity of the CYP3A4/5. In further aspects of the above embodiment, the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In further aspects of the above embodiment, the tritium-labeled testosterone is labeled at the 1, 2, 6β, and 7 positions. In a particularly preferred aspect of the above embodiments and aspects, the tritium-labeled testosterone is labeled solely at the 6β position.

The present invention further provides a method for identifying an analyte that inhibits activity of cytochrome 3A4 or 3A5 (CYP3A4/5), which comprises providing a multiwell plate and a column plate having an array of solid phase extraction cartridges or columns having therein a water-wettable polymer formed by copolymerizing at least one hydrophilic monomer and at least one lipophilic monomer in a ratio sufficient for the polymer to be water-wettable and effective at retaining organic solutes thereon; applying to each of the wells of the multiwell plate an aqueous mixture comprising CYP3A4/5, tritium-labeled testosterone labeled with tritium solely at the 6β position, and an analyte; contacting NADPH and optionally an NAPDH regenerating system to the aqueous mixture in each of the wells above and incubating for a time sufficient for the CYP3A4/5 to hydroxylate the tritium-labeled testosterone at the 6β position, which produces tritium-labeled water; optionally separating the CYP3A4/5 from the aqueous mixture in each of the wells of the multiwell plate; applying each aqueous mixture to a separate minicolumn of the column plate to remove the tritium-labeled testosterone from the aqueous mixture; and, measuring amount of the tritium in the aqueous mixture with the tritium-labeled testosterone removed wherein a decrease in the amount of the tritium in the presence of the analyte compared to the amount of the tritium in the absence of the analyte indicates that the analyte inhibits activity of the CYP3A4/5.

In further aspects of the above embodiment, the lipophilic monomer comprises a phenyl, phenylene, ether, or C₂-C₁₈ alkyl group. In a further still aspect, the lipophilic monomer is divinylbenzene.

In further aspects of the above embodiment, the hydrophilic monomer comprises a saturated, unsaturated, or aromatic heterocyclic group. In a further still aspect, the hydrophilic monomer is N-vinylpyrrolidone.

In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In further still aspects of any one of the above embodiments and aspects, each of the minicolumns of the column plate further comprises a porous retaining means for retaining the polymer therein. In a preferred embodiment, the wells of the multiwell plate and column plate each have a 96-well tissue culture plate format.

The present invention further provides a method for identifying an analyte that irreversibly inhibits activity of CYP3A4/5, which comprises providing a mixture comprising CYP3A4/5, NADPH regenerating system, and the analyte; incubating the mixture for different times; diluting the mixture and then adding to the diluted mixture tritium-labeled testosterone labeled with tritium at the 6, position and NADPH; incubating the diluted mixture for a time sufficient for the CYP3A4/5 to hydroxylate the tritium-labeled testosterone at the 6, position; removing the CYP3A4/5 from the mixture; applying the mixture to a sorbent which preferentially binds non-polar compounds to remove the tritium-labeled testosterone from the mixture; and measuring amount of the tritium in the mixture of step (d) with the tritium-labeled testosterone removed, wherein a decrease in the amount of the tritium indicates that the analyte irreversibly inhibits activity of the CYP3A4/5.

In a further aspect of the above embodiment, the sorbent comprises a water-wettable polymer formed by copolymerizing at least one hydrophilic monomer and at least one lipophilic monomer in a ratio sufficient for the polymer to be water-wettable and effective at retaining organic solutes thereon. In further aspects of the above embodiment, the lipophilic monomer comprises a phenyl, phenylene, ether, or C₂-C₁₈ alkyl group. In a further still aspect, the lipophilic monomer is divinylbenzene. In further aspects of the above embodiments, the hydrophilic monomer comprises a saturated, unsaturated, or aromatic heterocyclic group. In a further still aspect, the hydrophilic monomer is N-vinylpyrrolidone. In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In another aspect of the above embodiment, the sorbent comprises a non-polar group bonded to a silica substrate. In a further still aspect, the sorbent comprises one or more silanes selected from the group consisting phenyl silane, dimethylsilane, trimethylsilane, ethyl silane, butyl silane, hexyl silane, octyl silane, and octadecyl silane. In further still aspects, the silica substrate is selected from the group consisting of silica particles and silica gel.

In further aspects of the above embodiment, the tritium-labeled testosterone is labeled at the 1, 2, 6β, and 7 positions. In a particularly preferred aspect of the above embodiments and aspects, the tritium-labeled testosterone is labeled solely at the 6β position.

In particular embodiments of any one of the above embodiments and aspects, the CYP3A4/5 is provided in microsomes. The microsomes can be produced from cells selected from the group consisting of mammalian and insect cells, wherein the cells include a vector (e.g., viral or plasmid vectors) expressing the CYP3A4/5 or the microsomes can be from kidney, liver, brain, muscle, or the like cells. Preferably, the microsomes are human liver microsomes (HLM). In particular embodiments of any one of the above embodiments and aspects which use HLM as the source for CYP3A4/5, the HLM are removed from the aqueous mixture by acidification and/or centrifugation.

In a further embodiment, the present invention provides a method for determining the activity of CYP3A4/5 in hepatocytes, which comprises providing a culture of the hepatocytes; incubating the hepatocytes in a medium comprising testosterone labeled with tritium at the 6β position for a time sufficient for the CYP3A4/5 to hydroxylate the tritium-labeled testosterone at the 6β position, which produces tritium-labeled water; removing the medium from the culture of hepatocytes; applying the medium to a sorbent which preferentially binds non-polar compounds to remove the tritium-labeled testosterone from the medium; and measuring amount of the tritium in the medium with the tritium-labeled testosterone removed, which determines the relative activity of the CYP3A4/5 in the hepatocytes.

In a further still embodiment, the present invention provides a method for identifying an analyte that induces CYP3A4/5 expression, which comprises providing a culture of hepatocytes; incubating the hepatocytes in a medium comprising the analyte; replacing the medium comprising the analyte with a second medium comprising testosterone labeled with tritium at the 6β position and incubating the hepatocytes for a time sufficient for the CYP3A4/5 to hydroxylate the tritium-labeled testosterone at the 6β position, which produces tritium-labeled water; removing the second medium from the culture of hepatocytes; applying the second medium to a sorbent, which preferentially binds non-polar compounds, to remove the tritium-labeled testosterone from the second medium; and measuring amount of the tritium in the second medium with the tritium-labeled testosterone removed wherein an increase in the amount of tritium compared to a control culture of hepatocytes incubated with the tritium labeled testosterone and without the analyte indicates that the analyte induces CYP3A4/5 expression. Preferably, the hepatocytes are incubated in the medium comprising the analyte for between about 24 to 78 hours.

In a further embodiment, the present invention provides a method for identifying an analyte that inhibits CYP3A4/5 activity, which comprises providing a culture of hepatocytes; incubating the hepatocytes in a medium comprising testosterone labeled with tritium at the 6β position and the analyte for a time sufficient for the CYP3A4/5 to hydroxylate the tritium-labeled testosterone at the 6β position, which produces tritium-labeled water; removing the medium from the culture of hepatocytes; applying the medium to a sorbent, which preferentially binds non-polar compounds, to remove the tritium-labeled testosterone from the medium; and measuring amount of the tritium in the medium of step with the tritium-labeled testosterone removed wherein a decrease in the amount of tritium compared to a control culture of hepatocytes incubated with the tritium labeled testosterone and without the analyte indicates that the analyte inhibits the CYP3A4/5 activity.

In a further aspect of the above embodiments, the culture of hepatocytes is provided in one or more wells of a multiwell plate and the sorbent is provided packed in one or more solid phase extraction cartridges or columns comprising a column plate.

In a further aspect of the above embodiments, the sorbent comprises a water-wettable polymer formed by copolymerizing at least one hydrophilic monomer and at least one lipophilic monomer in a ratio sufficient for the polymer to be water-wettable and effective at retaining organic solutes thereon. In further aspects of the above embodiment, the lipophilic monomer comprises a phenyl, phenylene, ether, or C₂-C₁₈ alkyl group. In a further still aspect, the lipophilic monomer is divinylbenzene. In further aspects of the above embodiments, the hydrophilic monomer comprises a saturated, unsaturated, or aromatic heterocyclic group. In a further still aspect, the hydrophilic monomer is N-vinylpyrrolidone. In further still aspects of the above embodiment, the water wettable polymer is poly(vinylbenzene-co-N-vinylpyrrolidone, preferably, a polymer wherein the poly(vinylbenzene-co-N-vinylpyrrolidone comprises more than 12 mole percent N-vinylpyrrolidone, more preferably, a polymer wherein the poly(vinylbenzene-co-N-vinylpyrrolidone comprises from about 15 mole percent to about 30 mole percent N-vinylpyrrolidone.

In another aspect of the above embodiments, the sorbent comprises a non-polar group bonded to a silica substrate. In a further still aspect, the sorbent comprises one or more silanes selected from the group consisting phenyl silane, dimethylsilane, trimethylsilane, ethyl silane, butyl silane, hexyl silane, octyl silane, and octadecyl silane. In further still aspects, the silica substrate is selected from the group consisting of silica particles and silica gel.

In further aspects of the above embodiments, the tritium-labeled testosterone is labeled at the 1, 2, 60, and 7 positions. In a particularly preferred aspect of the above embodiments and aspects, the tritium-labeled testosterone is labeled solely at the 6β position. In a further still aspect, the testosterone is labeled solely at the 6β position by incubating in a reaction mixture testosterone-3-ethyleneacetal and peracetic acid; separating 5β,6β-epoxide (5β,6β-epoxy-17β-hydroxyandrostan-3-one,3-ethyleneacetal) from 5α,6α-epoxide (5α,6α-Epoxy-17β-hydroxyandrostan-3-one,3-ethyleneacetal) produced in the reaction mixture; incubating the 5α,6α-epoxide in tetrahedrofuran containing [³H]-lithium aluminum to produce [6β-³H]-3,3-ethylenedioxyandrostane-5α,17β-diol; and incubating the [6β-³H]-3,3-ethylenedioxyandrostane-5α,17β-diol in an aqueous mixture of acetic acid to produce the [6β-³H]-testosterone.

In further still embodiments of the above, the water wettable polymer is packed inside a solid phase extraction cartridge or column. In a particularly preferred embodiment of any one of the above, the method is performed in a multiwell plate format comprising a first multiwell plate for performing the incubation, a multicolumn plate in the same configuration as the multiwell plate for separating the labeled diclofenac from the tritiated water after the incubation, and a second multiwell plate for collecting the column void volume and washes from the multicolumn for determining the tritium therein.

As used herein, the term “tritium-labeled testosterone labeled with tritium at the 6β position” refers to testosterone labeled solely at the 6β position and testosterone labeled at the 1, 2, 6β, and 7 positions.

As used herein, the term “analyte” refers to molecules, compounds, chemicals, compositions, drugs, and the like.

### BRIEF DESCRIPTION OF THE DRAWINGS

FIG. 1 shows a cross-sectional view of an extraction cartridge or column 10.

FIG. 2 shows a perspective view of a multicolumn microfiltration/extraction plate 100.

FIG. 3A shows the dependence of [³H]-H₂O formation from [6β-³H]-testosterone on incubation time and concentration of human liver microsomes. Experiments were conducted in the presence of 10 μM unlabelled testosterone and the indicated concentrations of HLM. Product formation was expressed a percentage of total radioactivity. Each point is the mean ±SEM of duplicate determinations.

FIG. 3B shows the dependence of [³H]—H₂O formation from [6β-³H]-testosterone on incubation time and concentration of human liver microsomes. Experiments were conducted in the presence of 60 μM unlabelled testosterone and the indicated concentrations of HLM. Product formation was expressed a percentage of total radioactivity. Each point is the mean ±SEM of duplicate determinations.

FIG. 4A shows the effect of CYP inhibitors on [³H]-H₂O formation from [1,2,6,7-³H]-testosterone in human liver microsomes. Reactions were conducted in the presence of 60 μM unlabelled testosterone and in the presence or absence of the following compounds: 30 μM furafylline, 50 μM coumarin, 10 μM sulfaphenazole, 10 μM quinidine, 100 μM diethyldithiocarbamate, 1 μM ketoconazole. Enzyme activity is expressed as a percentage of untreated controls and represents the average z standard error of duplicate determinations.

FIG. 4B shows the effect of anti-CYP monoclonal antibodies on [³H]-H₂O formation from [1,2,6,7-³H]-testosterone in human liver microsomes. Reactions were conducted in the presence of 60 μM unlabelled testosterone and in the presence or absence of 0.4 mg/mL of antibodies. Enzyme activity is expressed as a percentage of untreated controls and represents the average ±standard error of duplicate determinations.

FIG. 5A shows the effect of unlabelled testosterone on [³H]-H₂O formation from [6β-³H]-testosterone in human liver microsomes. Testosterone was added from stock solutions in methanol, with a final methanol concentration of 0.6% in the assay. Product formation was assessed in the absence (filled symbols) or presence (empty symbols) of 10 μM ketoconazole. Results are average ±SEM from 3 separate experiments.

FIG. 5B shows the dependence of v′ on total substrate concentration. The term v′, defined as described in Example 1, was calculated from the product counts shown in FIG. 5A. Data were fitted to the Hill equation by nonlinear regression analysis.

FIG. 5C shows the dependence of the velocity of formation of 6β-hydroxytestosterone on total substrate concentration. Data are average ±SEM from 3 separate experiments.

FIG. 5D shows the effect of unlabelled testosterone on [³H]-H₂O formation from [1,2,6,7-³H]-testosterone in human liver microsomes. Product formation was assessed in the absence (filled symbols) or presence (empty symbols) of 10 μM ketoconazole. Results are average ±SEM, n=2.

FIG. 6A shows the effect of Ketoconazole on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 6B shows the effect of Nifedipine on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 6C shows the effect of Compound A on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 6D shows the effect of Miconazole on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 6E shows the effect of Bromocriptine on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 6F shows the effect of Nicardipine on [³H]-H₂O formation from [1,2,6,7-³H]-testosterone in human liver microsomes. Data were fitted to a four parameter logistic equation by nonlinear regression analysis. IC₅₀ values are summarized in Table 3. Data are average ±SEM from duplicate experiments.

FIG. 7A shows a comparison between radiometric and LC-MS/MS assays. IC₅₀ values for 29 Merck NCEs in the radiometric [6β-³H]-testosterone hydroxylation assay were correlated with those obtained in the conventional testosterone 6β-hydroxylation assay. Data from the conventional assay were on file at Merck Research Laboratories.

FIG. 7B shows a comparison between radiometric and LC-MS/MS assays. Correlation analysis was performed using data from 28 compounds, excluding the compound highlighted by an arrow in FIG. 7A.

FIG. 8A shows a comparison between the radiometric and LC-MS/MS assays to determine the effect of the CYP3A4 inducer rifampicin on testosterone 6β-hydroxylase activity in cultured human hepatocytes from donor 1.

FIG. 8B shows a comparison between the radiometric and LC-MS/MS assays to determine the effect of the CYP3A4 inducer rifampicin on testosterone 6β-hydroxylase activity in cultured human hepatocytes from donor 2.

FIG. 9A shows the time-dependent inhibition of CYP3A4/5 activity by mifepristone. CYP3A4/5 activity was determined by radiometric testosterone 6β-hydroxylase assay and shows the percent CYP3A4/5 activity remaining after different times of preincubation at the indicated concentrations of mifepristone.

FIG. 9B shows the fitting of the inactivation rate vs. inhibitor concentration curve to derive kinetic parameters of inhibition for the time-dependent inhibition of CYP3A4/5 activity by mifepristone as determined by radiometric testosterone 6β-hydroxylase assay. CYP3A4/5 activity was determined by radiometric testosterone 6β-hydroxylase assay.

FIG. 10A shows the time-dependent inhibition of CYP3A4/5 activity by mifepristone. CYP3A4/5 activity was determined by testosterone 6β-hydroxylase assay with LC-MS/MS detection and shows the percent CYP3A4/5 activity remaining after different times of preincubation at the indicated concentrations of mifepristone.

FIG. 10B, shows the fitting of the inactivation rate vs. inhibitor concentration curve to derive kinetic parameters of inhibition for the time-dependent inhibition of CYP3A4/5 activity by mifepristone. CYP3A4/5 activity was determined by testosterone 6β-hydroxylase assay with LC-MS/MS detection.

### DETAILED DESCRIPTION OF THE INVENTION

The present invention provides a rapid and sensitive testosterone 6β-hydroxylation assay for assessing cytochrome P-450 isoform 3A4 or 3A5 (CYP3A4/5) activity and for identifying modulators of CYP3A4 activity or expression. In particular, the present invention provides an assay for assessing the activity of CYP3A4/5 in mixtures comprising CYP3A4/5. The assays include both reversible inhibition assays and mechanism-based or time-dependent inhibition assays. Examples of mixtures include microsomes from various tissues such as human liver microsomes (HLM); microsomes from mammalian or insect cells containing an expression vector which expresses recombinant CYP3A4/5; or hepatocytes, the potential of an analyte to inhibit CYP3A4/5 activity in any of the above mixtures, and the potential of an analyte to induce CYP3A4/5 expression in hepatocytes. Preferably, the CYP3A4/5 is a human CYP3A4/5. The assay is based on detecting the release of tritium as [³H]-H₂O which occurs upon CYP3A4/5-mediated 6β-hydroxylation of testosterone labeled with tritium in the 6β position in the presence of the analyte wherein an increase or decrease in the release of the tritium over time indicates that the analyte is a modulator of CYP3A4/5 activity. For example, a decrease in the release of tritium in HLM in the presence of an analyte indicates that the analyte is an inhibitor of CYP3A4/5 activity whereas an increase in the release of tritium in hepatocytes after treatment of the hepatocytes with the an analyte indicates that the analyte is an inducer of CYP3A4/5 activity. The tritiated water product is separated from tritiated testosterone in a solid-phase extraction process using a sorbent a sorbent comprising a substrate which preferentially binds non-polar compounds such as testosterone. All the steps of the assay, including incubations, product separation, and radioactivity counting are performed in a multiwell format, which can be automated.

The embodiment for identifying analytes that induce or inhibit CYP3A4/5 activity using hepatocytes in one aspect identifies analytes that inhibit or induce expression of the gene encoding CYP3A4/5, i.e., analytes which affect transcription of the gene encoding CYP3A4/5. The embodiment in another aspect identifies analytes that exert their inhibitory or inducing effect on CYP3A4/5 activity by affecting posttranscriptional processing of mRNA encoding the CYP3A4/5. The embodiment in a further aspect identifies analytes that exert their inhibitory or inducing effect on CYP3A4/5 activity by affecting translation of the mRNA encoding the CYP3A4/5. The embodiment in a further still aspect identifies analytes that exert their inhibitory or inducing effect on CYP3A4/5 activity by interacting directly or indirectly with the CYP3A4/5.

The embodiment for assessing CYP3A4/5 activity is useful for controlling the activity of commercial batches of hepatocytes or the quality of hepatocytes isolated in house, for instance, before using these hepatocytes to perform metabolic stability studies with new chemical entities. The embodiment for identifying CYP3A4 modulators is useful for assessing the CYP3A4/5 inhibition or induction potential of drug candidates in order to exclude drug candidates that are potent inhibitors or inducers from further development. In either embodiment, the present invention is an improvement over assays of the prior art which rely on HPLC separation and mass spectrometry to assess the CYP3A4/5 inhibition or induction potential of an analyte.

While the assays are described herein using HLM or hepatocytes, the assays can use purified recombinant CYP3A4/5 or microsomes prepared from other tissues, for example, kidney, intestine, lung, or the like, or other subcellular fractions containing microsomes. The microsomes can be prepared from mammalian cells containing a plasmid or viral vector that expresses CYP3A4/5, preferably, a human CYP3A4/5. The microsomes can be from insect cells infected with recombinant baculovirus expressing CYP3A4/5 and a p450 reductase. The advantage of the cells expressing recombinant CYP3A4/5 is that CYP3A4/5 is the only cytochrome P450 present in these microsomes and the specific activity is generally higher. The concentration range for assays using recombinant CYP3A4 is from about 1 to 100 pmol/mL, preferred concentrations are between about 5 to 50 pmol/mL. For time-dependent assays, the enzyme should be 5-10-fold higher (because of the final dilution in the second incubation).

To test an analyte for inhibition of CYP3A4/5 activity, a first container is provided which contains an aqueous mixture comprising the analyte to be tested for an inhibitory effect on CYP3A4/5 activity, testosterone labeled with tritium at the 6β position as the substrate probe, unlabelled testosterone to provide an adequate concentration of substrate, pooled HLM, and a buffer at a physiological pH. Typically, between about 100,000 to 2,000,000 dpm of tritium labeled testosterone is used, preferably, the labeled testosterone is at about 1,000,000 dpm. In a preferred embodiment, the tritiated testosterone is labeled solely at the 6β position, which can be used at between about 100,000 to 200,000 dpm. The amount of unlabelled testosterone is between about 1 to 100 μM, typically at about 60 μM. The pooled HLM are generally at about 0.05 to 2 mg/mL, typically, about 0.25 mg/mL. An example of a suitable buffer is 0.1 M potassium phosphate, pH 7.6). The final volume is preferably between about 100 μL to 200 μL. Preferably, a control containing an equivalent amount of the vehicle used for the analyte is provided.

In general, while the assay can be performed using commercially available testosterone labeled with tritium at the 1, 2, 6, and 7 positions as the substrate probe, the preferred substrate probe is testosterone labeled with tritium solely at the 6β position. When tritiated testosterone labeled solely in the 6β position is used in the assay, both the fractional conversion rate and the sensitivity of the assay are improved. Furthermore, reactions using testosterone labeled sole in the 6β position can be performed in a volume of about 100 μL whereas reactions using commercially available testosterone labeled at the 1, 2, 6, and 7 positions are preferably performed in a volume of about 200 μL. Synthesis of [6β-³H]-testosterone has been described by Campbell et al. in J. Amer. Chem. Soc. 80: 4717-721-4721 (1958), Toft et al., in J. Labeled Compounds and Radiopharmaceuticals, 9: 413 (1973), and Liston and Toft in J. Org. Chem. 34: 2288 (1969) and is described in Example 1.

Following a preferred preincubation step of microsomes in buffer for several minutes at 37° C., about 1 mM NADPH with or without an NADPH regenerating system comprising about 5 mM glucose-6-phosphate, about 3 mM MgCl₂, and about 1 unit/mL glucose-6-phosphate dehydrogenase is added to the aqueous mixture to form a reaction mixture which is then incubated at 37° C. for a period of time sufficient to allow 6β hydroxylation of the testosterone. In general, about 10 minutes is usually sufficient to detect CYP3A4/5 activity. In some cases, a multiplicity of assays are performed for various lengths of time. The reaction mixture is then stopped by addition of an acid such as HCl at a concentration of about 0.1 N. Preferably, the HLM are removed from the aqueous mixture before transferring the reaction mixture to an extraction cartridge or column for separating tritiated water from the tritiated testosterone. The HLM can be removed from the aqueous layer by filtration, centrifugation, or the like. In a preferred embodiment, the HLM are removed by centrifugation. Because the acidification of the reaction causes the proteins in the HLM to precipitate, the proteins of the HLM can be removed using low speed centrifugation.

The aqueous mixture with the HLM removed or the reaction mixture containing the ELM is transferred to an extraction cartridge or column containing a sorbent which preferentially binds non-polar compounds such as testosterone. The void volume or flow-through from the column is collected in a second container. The sorbent in the column is washed with water and the washes transferred to the second container. Scintillation fluid is added to the second container and the tritium released from the tritiated testosterone by CYP3A4/5 is measured. Alternatively, the void volume or flow-through and washes are transferred to a scintillation vial and mixed with scintillation fluid for measuring the tritium in a scintillation counter. The absence of tritiated water or reduced amounts of tritiated water compared to the amounts of tritiated water in the positive controls indicate that the analyte is an inhibitor of CYP3A4/5 activity.

The CYP3A4/5 activity of a preparation of hepatocytes from liver tissue is determined as follows. Primary cultures of hepatocytes, which can comprise hepatocytes freshly isolated from liver tissue or which had been isolated previously, frozen for storage, and thawed for the assay, are provided. The hepatocytes are maintained at 37° C. in a humidified atmosphere of 5% CO₂ and 95% air or oxygen in a culture medium or aqueous mixture suitable for culturing hepatocytes (See for example, Dich and Grunnet in Methods in Molecular Biology, Vol. 5: Animal Cell Culture (Pollard, and Walker, eds) pp. 161-176, Humana Press, Clifton, N.J. (1989). The assay can be performed using either cells in suspension or cultured cells attached to cell culture plates. For suspension assays, typically, the hepatocytes are incubated at a concentration of about 1×10⁵ cells/mL to 1×10⁶ cells/mL, preferably 1×10⁶ cells/mL. Thus, each culture well contains about 1×10⁶ cells, 1 mL of hepatocyte culture medium (HCM) (Dich and Grunnet, ibid.), unlabelled testosterone, and tritium-labeled testosterone. Typically, between about 100,000 to 2,000,000 dpm of tritium labeled testosterone is used. In a preferred embodiment, the tritiated testosterone is labeled solely at the 6, position, which can be between about 100,000 to 500,000 dpm. The amount of unlabelled testosterone is between about 1 to 200 μM, typically at about 60 to 200 μM. For assays in plated cells, the hepatocytes are plated onto tissue culture plates (preferably, the culture plates are collagen-coated 24- or 96-well tissue culture plates) and maintained at 37° C. in a humidified atmosphere of 5% CO₂ in a culture medium suitable for culturing fresh hepatocytes, e.g., HCM. Preferably, the medium is supplemented with ITS. Typically, the hepatocytes are plated at a density of about 150,000 to 200,000 cells/cm².

Following the incubation, the incubation medium is removed from the cells, for instance by centrifugation, and transferred to an extraction cartridge or column containing a sorbent which preferentially binds non-polar compounds such as testosterone. The void volume or flow-through from the column is collected in a second container. The sorbent in the column is washed several times with water and the washes transferred to the second container. Scintillation fluid is added to the second container and the tritium released from the tritiated testosterone by CYP3A4/5 is measured. Alternatively, the void volume or flow-through and washes are transferred to a scintillation vial and mixed with scintillation fluid for measuring the tritium in a scintillation counter. The amounts of tritiated water produced determines the relative CYP3A4/5 activity of the hepatocytes.

The assay for determining the ability of an analyte to inhibit CYP3A4/5 activity is as follows. Primary cultures of hepatocytes, which can comprise hepatocytes freshly isolated from liver tissue or which had been isolated previously, frozen for storage, and thawed for the assay, are provided. The assay can be performed using either cells in suspension or cultured cells attached to cell culture plates. For suspension assays, the hepatocytes are maintained at 37° C. in a humidified atmosphere of 5% CO₂ in a culture medium suitable for culturing hepatocytes as above. Typically, the hepatocytes are incubated at a concentration of about 1×10⁶ cells/mL. For non-suspension assays, the hepatocytes are plated to collagen-coated plates and maintained at 37° C. in a humidified atmosphere of 5% CO₂ in a culture medium suitable for culturing hepatocytes, e.g., HCM. Thus, each culture well contains about 1×10⁶ cells, 1 mL of HCM, the analyte being tested for inhibitory effect on CYP3A4/5 activity, unlabelled testosterone, and tritium-labeled testosterone. Typically, between about 100,000 to 2,000,000 dpm of tritium labeled testosterone is used. In a preferred embodiment, the tritiated testosterone is labeled solely at the 6β position, which can be between about 100,000 to 500,000 dpm. The amount of unlabelled testosterone is between about 1 to 200 μM, typically at about 60 to 200 μM. Preferably, controls that include the vehicle for the analyte or a CYP3A4 inhibitor such as ketoconazole are provided.

Following the incubation, the incubation medium is removed from the cells and transferred to an extraction cartridge or column containing a sorbent which preferentially binds non-polar compounds such as testosterone. The void volume or flow-through from the column is collected in a second container. The sorbent in the column is washed several times with water and the washes transferred to the second container. Scintillation fluid is added to the second container and the tritium released from the tritiated testosterone by CYP3A4/5 is measured. Alternatively, the void volume or flow-through and washes are transferred to a scintillation vial and mixed with scintillation fluid for measuring the tritium in a scintillation counter. The absence of tritiated water or reduced amounts of tritiated water compared to the amounts of tritiated water in the control comprising the vehicle only indicates that the analyte is an inhibitor of CYP3A4/5 activity.

The assay for determining the ability of an analyte to induce CYP3A4/5 activity is as follows. Primary cultures of hepatocytes, which can comprise hepatocytes freshly isolated from liver tissue or which had been isolated previously, frozen for storage, and thawed for the assay, are provided. The hepatocytes are plated onto tissue culture plates (preferably, the culture plates are collagen-coated 24- or 96-well tissue culture plates) and maintained at 37° C. in a humidified atmosphere of 5% CO₂ in a culture medium suitable for culturing fresh hepatocytes, e.g., HCM. Preferably, the medium is supplemented with ITS. Typically, the hepatocytes are plated at a density of about 150,000 to 200,000 cells/cm². Twenty-four to 78 hours later, the culture medium is removed and fresh medium and the analyte to be tested for induction potential are added to the hepatocytes. Preferably, controls are provided which comprise either the vehicle for the analyte or a known inducer such as Rifampicin. After incubating the hepatocytes as above for time sufficient for induction of CYP3A4/5, usually between about 24 to 78 hours, CYP3A4/5 enzyme activity is determined.

The hepatocytes are incubated in an incubation medium containing a balanced salt solution containing a buffer at physiological pH, for example, pH 7.4. An example of a balanced salt solution is Hank's balanced salt solution and an example of a suitable buffer is 10 mM HEPES. Then a mixture containing unlabelled testosterone and tritium-labeled testosterone is added and the hepatocytes incubated as above for a suitable time to assess CYP3A4/5 activity, about an hour is usually sufficient. Typically, between about 100,000 to 2,000,000 dpm/mL of tritium labeled testosterone is used, preferably, the labeled testosterone is at about 1,000,000 dpm/mL. In a preferred embodiment, the tritiated testosterone is labeled solely at the 6, position, which can be used at between about 100,000 to 500,000 dpm/mL. The amount of unlabelled testosterone is between about 1 to 300 μM, typically at about 60 μM. Optionally, parallel incubations are performed, which contain the CYP3A4 inhibitor ketoconazole, to ascertain that detected enzyme activity is specifically mediated by CYP3A4/5.

Following the incubation, the incubation medium is removed from the cells and transferred to an extraction cartridge or column containing a sorbent which preferentially binds non-polar compounds such as testosterone. The void volume or flow-through from the column is collected in a second container. The sorbent in the column is washed with water and the washes transferred to the second container. Scintillation fluid is added to the second container and the tritium released from the tritiated testosterone by CYP3A4/5 is measured. Alternatively, the void volume or flow-through and washes are transferred to a scintillation vial and mixed with scintillation fluid for measuring the tritium in a scintillation counter. The presence of tritiated water or increased amounts of tritiated water compared to the amounts of tritiated water in the control with the vehicle only indicates that the analyte is an inducer of CYP3A4/5 activity.

As discussed below and shown in Example 1, in a preferred aspect of the present invention, the assay is performed in a multiwell format, preferably, a 96-well format. The multiwell format enables a plurality of analytes to be tested simultaneously. In the multiwell format, each reaction is conducted in the well of a multiwell plate (first container). The separation of tritiated water from tritiated testosterone at the conclusion of the reaction and following the optional step of removing the ELM is performed by applying each reaction to a separate column of a microfiltration/extraction column plate comprising a plurality of miniature columns, each containing the sorbent disclosed herein. Preferably, the columns of the microfiltration/extraction column plate are arranged in the same format as the format for the multiwell plate. The void volume and washes are collected in a second multiwell plate in the same format as the microfiltration/extraction column plate, mixed with scintillation fluid, and counted in a scintillation counter adapted for counting samples in a multiwell format.

The sorbent preferentially binds non-polar compounds such as testosterone, i.e., the sorbent can adsorb or bind the labeled testosterone but not the labeled water produced by the hydroxylation. Sorbents which preferentially bind non-polar compounds such as testosterone include, but are not limited to, sorbents comprising a hydrophobic or lipophilic polymer such as polystrene-divinylbenzene or poly(divinyl-benzene-vinylpyrrolidone), water-wettable polymers comprising lipophilic and hydrophilic monomers in a ratio that enables the sorbent to bind the labeled testosterone but not tritiated water, and silicon-based sorbents such as the C₂-C₁₈ silanes.

The sorbent comprising a water-wettable polymer is formed by copolymerizing at least one hydrophilic monomer and at least one lipophilic monomer in a ratio sufficient for the polymer to be water-wettable and effective at retaining organic solutes thereon. The lipophilic monomer can comprise a lipophilic moiety such as phenyl, phenylene, and C₂-C₁₈-alkyl groups. Particularly useful lipophilic monomers include divinylbenzene and styrene. The hydrophilic monomer can comprise a hydrophilic moiety such as a saturated, unsaturated, or aromatic heterocyclic groups, for example, a pyrrolidonyl group or a pyridyl group. Alternatively, the hydrophilic group can be an ether group. Particularly useful monomers include N-vinylpyrrolidone, 2-vinylpyridine, 3-vinylpyridine, 4-vinylpyridine, and ethylene oxide. In one embodiment of the water-wettable polymer, the polymer is a poly(divinylbenzene-co-N-vinylpyrrolidone) copolymer comprising greater than about 12 mole percent N-vinylpyrrolidone, preferably, from about 15 mole percent to about 30 mole percent N-vinylpyrrolidone. Examples of preferred water wettable polymers are disclosed in WO9738774 and U.S. Pat. No. 6,726,842, both to Bouvier et al. A preferred sorbent is the OASIS HLB sorbent, which comprises a balanced ratio of N-vinylpyrrolidone and divinylbenzene monomers, and is commercially available from Waters Corporation (Newcastle, Del.).

Sorbents comprising a silicon-based substrate or matrix include a non-polar group bonded to a silica substrate. The sorbent can comprise one or more silanes well known in the art for extracting non-polar compounds. Such sorbents include, but are not limited to, phenyl silane, butyldimethyl silane, dimethylsilane, trimethylsilane, ethyl silane, butyl silane, hexyl silane, octyl silane, or octadecyl silane. The silanes can be monofunctional or trifunctional. The silica substrate or matrix includes, but is not limited to, solid or porous silica or ceramic particles or microparticles or silica gel.

In a preferred embodiment of the method, the sorbent is provided as particles, beads, or the like are packed within an open-ended container to form a solid phase extraction cartridge or column. In particular embodiments of the method, the sorbent is packed into the solid phase extraction cartridge or column enmeshed in a porous membrane. In other embodiments, the solid phase extraction cartridge or column further includes a porous retaining means, such as a filter element, or frit at or near one or both ends of the solid phase extraction cartridge or column adjacent to the sorbent. The porous retaining means is to retain the sorbent within the solid phase extraction cartridge or column. In a further embodiment, the sorbent is disposed between a pair of porous retaining means, the first porous retaining means to retain the sorbent within the solid phase extraction cartridge or column and the second retaining means also aids in retaining the sorbent within the column and to prevent solid materials such as HLM from mixing with the sorbent. The filter or frit can be, for example, fritted glass, or a porous polymer such as high density polyethylene, TEFLON (E.I. du Pont de Nemours and Company, DE), or polycarbonate.

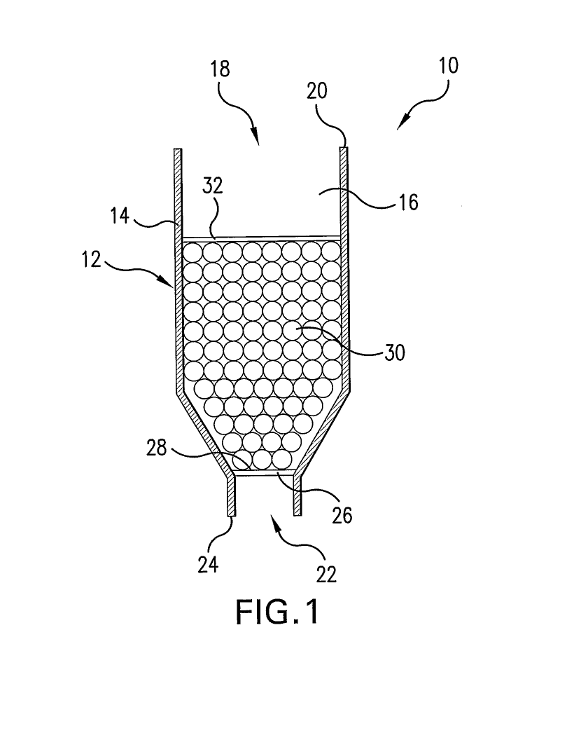

FIG. 1 shows a cross-sectional view of an example of a solid phase extraction cartridge or column 10 which is suitable for practicing the method of the present invention. The column 10 comprises an elongated body 12 having wall 14, which defines an axial hollow portion 16, an inlet 18 at the distal end 20 of the column 10 for receiving an aqueous mixture, and outlet 22 at the proximal end 24 of the column 10 for exit of the aqueous mixture. As further shown in FIG. 1, adjacent to the proximal end 24 is a porous retaining means 26 which has surface 28. The porous retaining means 26 is positioned adjacent to the proximal end 24 in column 10 so that surface 28 is perpendicular to wall 14 of column 10. Disposed on surface 28 of the porous retaining means 26 is sorbent 30. Optionally, as shown, a second porous retaining means 32 can be positioned adjacent to or near the distal end 20 and the sorbent 30 disposed therebetween. The column 10 enables the aqueous mixture to enter the container through the inlet 18, contact the sorbent 30 within the column 10, and exit the column 10 through the outlet 22. Preferably, the sorbent 30 is packed in the column 10 as small particles such as beads having a diameter preferably between about 30 to 60 μm.

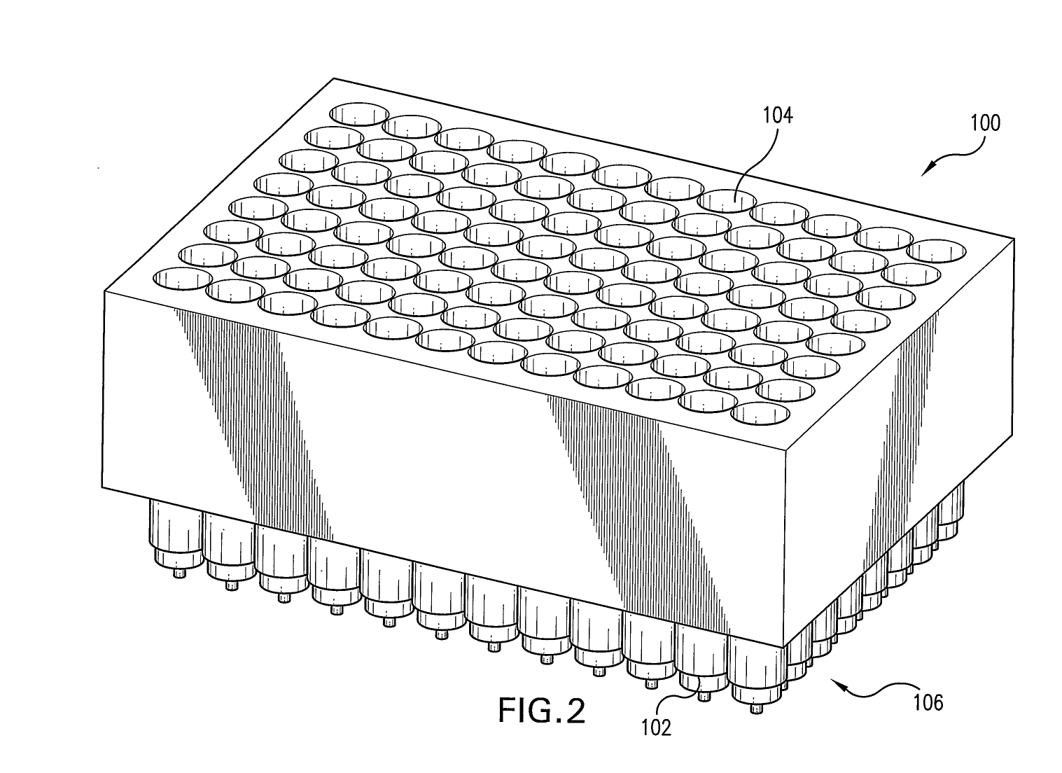

In a preferred embodiment, a multiplicity of the columns 10 are arranged to provide a format which is particularly suitable for high throughput screening. For example, a multicolumn microfiltration/extraction column plate comprising a multiplicity of wells adapted to provide solid phase extraction cartridges or columns (preferably, miniature solid phase extraction cartridges or columns, i.e., minicolumns). A preferred multicolumn microfiltration/extraction column plate format has the minicolumns arranged in a format that corresponds to the format used for multiwell tissue culture plates. For example, the minicolumns of the microfiltration/extraction column plate can be arranged in a 6-well, 12-well, 24-well, 48-well, 96-well, or 384-well format. In a preferred embodiment, the multicolumn microfiltration/extraction column plate has the minicolumns arranged in a 96-well format. As an example, FIG. 2 shows a multicolumn microfiltration/extraction plate 100 comprising a multiplicity of minicolumns 102 with opening 104 for receiving an aqueous mixture and outlet 106 for exit of the aqueous mixture wherein each of the minicolumns 102 comprises an internal arrangement similar to that shown for column 10 of FIG. 2 arrayed in a 96-minicolumn format. Movement of the aqueous mixture through the column and into a collecting plate containing wells arranged in a 96-well format can be achieved by centrifugation or by vacuum. Multi-column microfiltration/extraction column plates and methods and apparatus for using the plates have been disclosed in a number of U.S. Patents, for example, U.S. Pat. No. 6,506,343 to Bodner et al., U.S. Pat. No. 6,491,873 to Roberts and Woelk, and U.S. Pat. No. 6,338,802 to Bodner et al., and U.S. Published Patent Application No. 20030143124 to Roberts and Grenz.

In addition to reversible inhibition of CYP, irreversible or quasi-irreversible inactivation by certain analytes or their CYP-generated metabolites can occur. This type of inhibition, termed mechanism-based or time-dependent inhibition (MBI), is characterized by a progressive time-dependent decrease in enzyme activity in the presence of inhibitor. Three types of mechanism-based (time-dependent) inactivation of CYP have been reported: (i) inhibitor covalently binds to enzyme apoprotein; (ii) inhibitor covalently binds to prosthetic heme; (iii) inhibitor tightly (quasi-irreversibly) binds to heme or apoprotein. Most human hepatic drug-metabolizing CYPs, including CYP3A4/5, CYP2C9, CYP1A2, CYP2D6, CYP2C19, CYP2A6, CYP2B6 and CYP2E1 are subject to mechanism-based inhibition (MBI) (Zhang and Wong, Curr. Drug Metab. 6: 241-257 (2005); Venkatakrishnan et al., Curr. Drug Metab. 4: 423-459 (2003); Zhou et al., Curr. Drug Metab. 5: 415-442 (2004); Zhou et al., Clin. Pharmacokinet. 44: 279-304 (2005)).

In contrast to reversible CYP inhibition, whose effects are not always manifest in vivo, MBI almost invariably leads to clinically relevant drug-drug interactions. Indeed, it is currently thought that MBI might be one of the major causes for clinical drug-drug interactions, which has been potentially overlooked in the past.

Since MBI leads to a time-dependent loss of active enzyme, the clinical effects of a time-dependent CYP inhibitor on the pharmacokinetics of a drug that is metabolized by the same CYP is as follows:
MBI causes non-stationary PK upon multiple dosing
The extent of drug-drug interaction is time-dependent in onset and offset
High concentrations of inhibitor in intestinal lumen will cause significant effects on substrates whose oral bioavailability is limited by intestinal metabolism.
   CYP3A4 is one of the major hepatic and intestinal CYPs and is involved in the metabolism of more than 50% of clinically used drugs. Time-dependent inhibitors of CYP3A4 include the calcium channel blockers verapamil, nicardipine, diltiazem and mibefradil; the antiprogestinic agent mifepristone; the macrolide antibiotics troleandomycin, erythromycin and clarithromycin; and the HIV protease inhibitors ritonavir and nelfinavir.

Therefore, the present invention also provides mechanism-based or time-dependent assays in addition to the reversible or quasi-reversible assays described above. To assess the potential of a compound to act as a time-dependent CYP inhibitor, the analyte is preincubated with CYP3A4/5 in the presence of an NADPH regenerating system for a series of different lengths of time (typically from 0 minutes to 60 minutes). In general, CYP3A4/5 is provided at an amount about 5 to 10 times greater than the amount used in the reversible inhibition assays. Control incubations are performed in the absence of inhibitor to monitor for losses in enzyme activity due to thermal instability. At the end of the preincubation, the change in the amount of enzymatically active CYP relative to the time 0 preincubation time control is determined. This is achieved by performing a second incubation in which the preincubation is diluted about 10-fold and substrate is added. Enzyme activity is determined by measuring the amount of product formed during a specified time interval. Typical substrates used for time-dependent CYP inhibition assays are the same as those used for reversible inhibition assays above. For example, the Kₘ for CYP3A4/5 with testosterone is about 50 μM and the preferred concentration of testosterone is between about 200 to 500 μM. Example 4 provides an example of a time dependent assay using HLM.

In order to minimize any reversible CYP inhibition effect caused by the test analyte in the second incubation, the preincubation mixture is diluted several-fold (typically 5-20 times), the CYP substrate is added at a concentration several times (typically 5-10 times) higher than the concentration required for half-maximal activity (to minimize competitive inhibition by test compound), and the incubation time is short (typically 10 min). If an analyte acts as a time-dependent inhibitor, preincubation with CYP will cause a loss of enzyme activity with pseudo-first order kinetics. For each inhibitor concentration, the percentage of remaining enzyme activity (relative to a control without inhibitor) will change with time according to the equation:

% of remaining enzyme activity=100×e^{(-k×t)} equation 1

where k is the observed pseudo-first order inactivation rate constant, which is related to the inhibitor concentration during preincubation according to the following relationship:

$$k = \frac{{k}_{inact} \times {I}^{n}}{{K}_{0.5}^{n} + {I}^{n}} \qquad equation 2$$

where I is the inhibitor concentration, k_{inact} is the maximal inactivation rate constant, K_{0.5} is the inhibitor concentration at 50% k_{inact}, and n is the Hill coefficient. To determine k_{inact} and K_{0.5}, the curve of k versus I is fitted to equation 2 using non-linear regression analysis.

The following example is intended to promote a further understanding of the present invention.

### EXAMPLE 1

This example illustrates the development of the assay of the present invention and its use to identify inhibitors of CYP3A4/5 activity.

Synthesis of [6β-³H]-testosterone. Synthesis of [6β-³H]-testosterone was as follows.

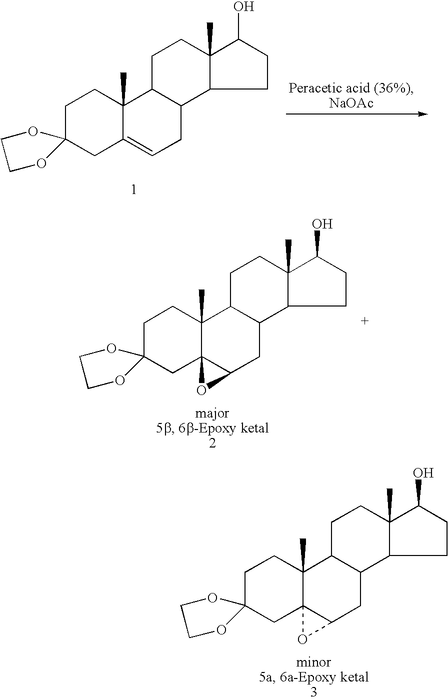

To a stirring solution of 0.85 g of testosterone-3-ethyleneacetal (1, obtained from Steraloids Inc., Newport, R.I.) in 14 mL of chloroform was added 0.1 g of sodium acetate and 1 mL of peracetic acid (36%) with cooling in an ice-salt bath. After two hours, the solution was washed first with 1N sodium hydroxide solution (5 mL) and then water (2×5 mL). The organic layer was dried over anhydrous magnesium sulfate, filtered, and concentrated to dryness. The 5β,6β-epoxide (5β,6β-epoxy-17β-hydroxyandrostan-3-one,3-ethyleneacetal; 2) and 5α,6α-epoxide (5α,6α-Epoxy-17β-hydroxyandrostan-3-one,3-ethyleneacetal; 3) which were formed in the reaction were separated from each other by using silica gel column chromatography (60 g. silica gel, hexane: acetone as an eluents). The fractions containing the 5α,6α-epoxide (3) were collected and evaporated to dryness to yield 330 mg of product. LC/MS: (MH)⁺349 (100%). ¹HNMR (CDCl₃) See Liston and Toft, ibid)

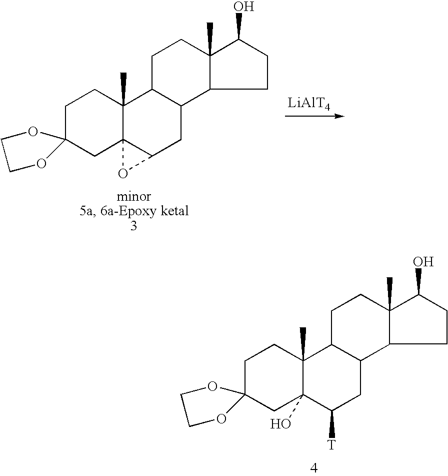

The 5α,6α-epoxy (3, 3.48 mg) in dry tetrahedrofuran (2 mL) was heated under reflux with stirring under nitrogen atmosphere with [³H]-lithium aluminum hydride (LAT, 100 mCi, obtained from American Radiolabelled Chemicals Inc., Saint Louis, Mo.) for one hour to produce [6β-³H]-3,3-ethylenedioxyandrostane-5α,17β-diol (4). The reaction mixture was quenched with 1% aqueous sodium hydroxide and diluted with ether (10 mL), washed with water (2×5 mL), and dried over anhydrous sodium sulfate. The solvent was evaporated to dryness and the residue used without further purification to the next reaction (total radioactivity 20 mCi).

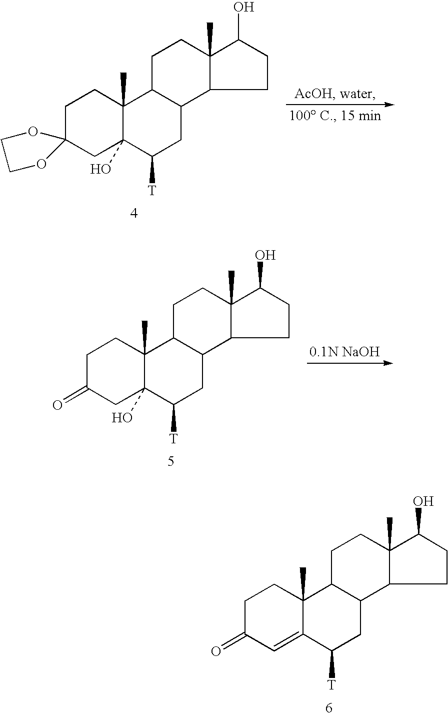

The [6β-³H]-3,3-ethylenedioxyandrostane-5α,17β-diol (4, 20 mCi) was heated at 100° C. for 15 minutes with a mixture of acetic acid (0.150 mL) and water (0.05 mL). The solution was cooled to room temperature and extracted with ether (2×10 mL). The organic layer was washed with saturated sodium bicarbonate (5 mL), water (2×5 mL) and dried over anhydrous sodium sulfate. The crude product (5, 10 mCi) was dissolved in methanol (5 mL), water (2 mL) and treated with 0.1N sodium hydroxide (0.1 mL). The solution was stirred at room temperature for 16 hrs and then treated with acetic acid (0.1 ml). The solution was evaporated to half volume on a rotary evaporator. The crude product was purified by reversed phase HPLC (Luna phenyl hexyl column, water containing 0.1% TFA: acetonitrile, 55:45, UV was 254 nm, flow rate 4 mL/min), retention time was 10.5 min). The combined fractions were passed through Sep-Pak C18, which was further washed with ethanol (10 mL), to yield 2.2 mCi of [6β-³H]-testosterone (6). The specific activity was 1.6 Ci/mmol as calculated by LC/MS. Tritium NMR confirmed the position of tritium labeled to be at 6β position of testosterone. LC/MS 289 (MH)+, 290 (MH+1)⁺, 291 (MH+2)⁺. Proton NMR: (CDCl₃, 8) 0.79 (3H, s, 18-H3), 1.2 (3H, s, 18-H3), 5.73 (1H, s, 4-H). Tritium NMR: (CDCl₃, 8) 2.1 (1 T, s, 6β-tritium).

Purification of [1,2,6,7-³H]-testosterone. (1,2,6,7-³H-testosterone was purchased from Amersham Biosciences (Piscataway, N.J.). To remove polar impurities, an aliquot (about 0.1 mCi) of commercially available [1,2,6,7-³H]-testosterone was dried under vacuum, reconstituted in water, and loaded on a 30-mg Waters OASIS extraction cartridge (preconditioned according to the manufacturer's instructions). OASIS extraction cartridges were purchased from Waters Corp. (Newcastle, Del.). Following washing with 5 mL of water, [1,2,6,7-³H]-testosterone was eluted with 2 mL of methanol, dried again, and reconstituted in ethanol (same volume as the original aliquot).

Radiometric CYP3A4 assays using [1,2,6,7-³H]-testosterone. Reactions were carried out in 96-well conical microtiter plates (available from Corning, Acton, Mass.) containing purified [1,2,6,7-³H]-testosterone tracer (0.2 to 0.5 μCi), unlabelled testosterone (60 μM, except otherwise noted), pooled human liver microsomes (ELM) (0.25 mg/mL, except otherwise noted), and 0.1 M potassium phosphate buffer, pH 7.6, in a final volume of 200 μL. Pooled HLM were obtained from Gentest Corp. (Woburn, Mass.). Inhibitors were added to the reaction mixture from stock solutions in DMSO, giving a final solvent concentration of 0.6% (v/v). No inhibitor controls contained an equivalent amount of vehicle. Following preincubation for 10 minutes at 37° C., reactions were started by addition of 1 mM NADPH and an NADPH regenerating system containing 5 mM glucose-6-phosphate, 3 mM MgCl₂, and 1 U/mL glucose-6-phosphate dehydrogenase. Assays were conducted for 10 minutes at 37° C. and stopped by addition of HCl to a final concentration of 0.1 N. Plates were then centrifuged for 10 minutes in a microplate rotor and supernatants loaded into the wells of a preconditioned 30 mg OASIS 96-well HLB plate. OASIS HLB 96 well extraction plates containing 10 or 30 mg of OASIS sorbent and vacuum manifold were purchased from Waters Corp. Vacuum was applied and the void volume collected in the collection plate. Then, 200 μL of water was added, vacuum was applied again, and the wash was collected into the same plate. This step was repeated. Pooled void volume and water washes were transferred into scintillation vials and counted in a beta-scintillation counter. Alternatively, aliquots were counted in 24-well scintillation plates using a TOPCOUNT scintillation counter (Packard, Perkin Elmer, Boston, Mass.). For the calculation of enzyme activity, product counts were corrected by subtraction of counts obtained in control incubations performed in the absence of NADPH regenerating system.

Radiometric CYP3A4 assays using [6β-³H]-testosterone. The assay procedure was similar to that of the [1,2,6,7-³H]-testosterone hydroxylation assay, with the following modifications. Total radioactivity was 100,000 to 200,000 dpm, reaction volume was 100 μL, and solvent concentration (coming from inhibitor stock solutions) was 0.3-0.7% DMSO and 0.5% acetonitrile (v/v). Product separation was carried out using 10 mg OASIS 96-well HLB plates. The void volume was collected and combined with a 75 μL water wash. Radioactivity was determined using a Beckman beta scintillation counter, or by counting 120 μL aliquots in 96-well scintillation plates using a TOPCOUNT scintillation counter.

Quantification of 6β-hydroxytestosterone and 2β-hydroxytestosterone. Aliquots of the assay reaction mixture and of metabolite standard curves were analyzed by HPLC using an Agilent HP1100 liquid chromatograph (Agilent Technologies) equipped with a CTC Analytics PAL autosampler (HTS PAL; CTC Analytics AG, Switzerland). Chromatography was performed on an XTERRA MS C18 column (4.6 mm×5 cm; 5 μm; Waters Corp.) at a flow rate of 1 mL/min, using a mobile phase consisting of a mixture of 0.1% formic acid in water (A) and 0.1% formic acid in acetonitrile (B) (linear gradient for 4 min from 5-100% B; 2 min at 100% B). The eluant was diverted to waste for the first minute, and then to a Sciex API-2000 triple quadrupole mass spectrometer with a Turbo Ionspray ionization source operated in the positive ion mode. The spray voltage was 5500V, the source temperature was 450° C., curtain gas setting was 10, declustering potential was 60V, focusing potential was 250V, and collision energy was 25 V. Metabolites were detected and identified using the transitions m/z 305.0→269.1. Metabolite concentrations were determined by weighted linear least-squares regression analysis of peak area ratios vs. those of a standard curve, using Analyst Quantitation Wizard software version 1.4.

Determination of the kinetic tritium isotope effect. TV/K, the kinetic isotope effect on the V/K ratio, was determined according to the formula (Northrop, Meth. Enzymol. 87: 607-625 (1982))

$${}^{T} V / K = \frac{log ( 1 - f )}{log ( 1 - f \frac{{SA}_{P}}{{SA}_{0}} )} \qquad equation 3$$

where f is the fractional conversion of substrate to product, SA₀ is the initial specific radioactivity of labeled substrate, and SA_{P} is the specific radioactivity of product. At low values of f (<5%), such as those observed in the present experiments, this expression reduces to (Northrop, Meth. Enzymol. 87: 607-625 (1982)):

$${}^{T} V / K \approx \frac{{SA}_{0}}{{SA}_{P}} \qquad equation 4$$

Calculation of the apparent rate of formation of unlabelled product from tracer competition experiments. When assays are performed using a fixed amount of [6β-³H]-testosterone and varying concentrations of unlabelled testosterone, the velocity of formation of unlabelled product, v, is given by:

v=v*/SAp equation 5

where v* is the velocity of formation of tritiated water.
Substituting from equation 4, we obtain:

v=v*x(^{T}V/K)/SA₀ equation 6

Defining v′ as the velocity of formation of unlabelled product divided by the kinetic isotope effect, i.e.

v′=v/(^{T}V/K) equation 7

we obtain

v′=v*/SA₀ equation 8

Without using the known ^{T}V/K ratio (which would be tautological, since it was derived from SA_{P}), v′, the apparent formation rate of unlabelled product, can be calculated. When plotted against the substrate concentration, S, and fitted to the Hill equation (equation 9), V′ₘₐₓ, S₅₀, the substrate concentration at 50%, and n, the Hill coefficient, can be derived.

$${v}^{′} = \frac{{V}_{max}^{′} \times {S}^{n}}{{S}_{50}^{n} + {S}^{n}} \qquad equation 9$$

where
V′ₘₐₓ=Vₘₐₓ/(^{T}V/K), i.e. the apparent maximal rate of product formation.

Curve fitting. Curve fitting to the Hill equation or to a four-parameter logistic inhibition model (Rodbard and Frazier, Meth. Enzymol. 37: 3-22 (1975)) was performed by nonlinear regression using XLFIT 4.0 (ID Business Solutions, Inc., Guildford, UK; Emeryville, Calif.).

### Results

Separation of radiolabelled testosterone and [3H]-H₂O using 96-well solid phase extraction plates. When a solution of assay buffer containing purified [1,2,6,7-³H]-testosterone (from 104 to 107 dpm) and stopping solution was applied to 96-well extraction plates containing 10 mg or 30 mg OASIS sorbent, over 99.9% of the radioactivity was retained on the plate. Only 0.045±0.006% (average ±SEM, n=7) of radioactivity eluted in the combined void volume and aqueous wash. For [6β-³H]-testosterone, 0.13±0.1% (average ±SEM, n=3) of radioactivity eluted in the aqueous fraction. The labeled testosterone could be recovered by eluting with methanol. In contrast to the labeled testosterone, [³H]-H₂O (from 102 to 105 dpm) was not retained under the same conditions. With both the 10 mg and 30 mg plates, recovery of [³H]-H₂O eluted in the combined void volume following a 100 μL or 400 μL aqueous wash, was quantitative (94±6%, average ±SD, n=6). Recoveries of radiolabelled testosterone and water were not affected by the presence of unlabelled testosterone at concentrations up to 600 μM. For chronological reasons, experiments with [1,2,6,7-³H]-testosterone and [6β-³H]-testosterone were performed using 30 mg and 10 mg OASIS plates, respectively.

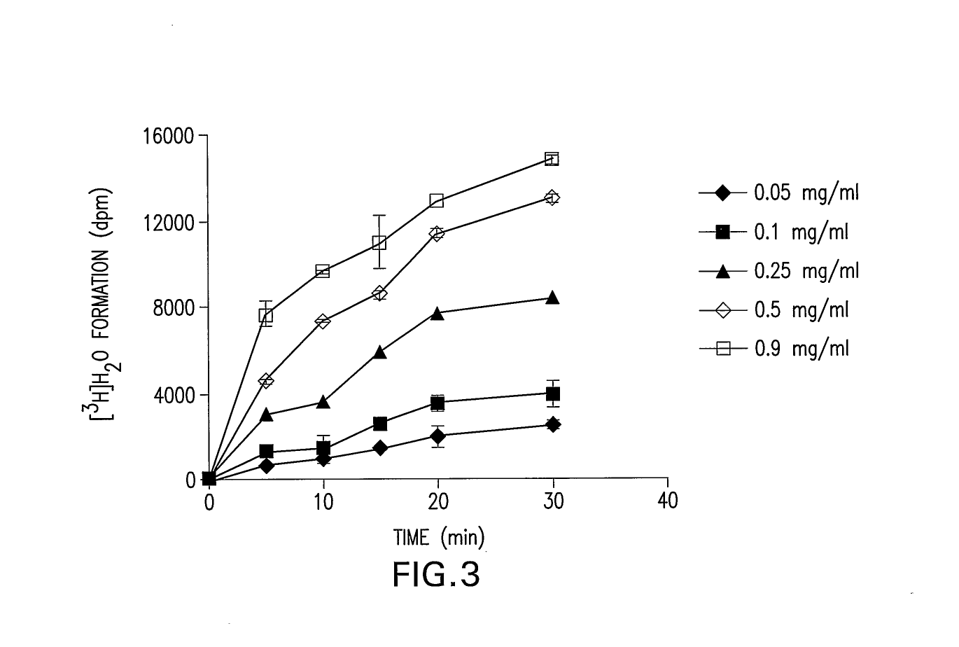

Formation of [³H]-H₂O from [1,2,6,7-³H]-testosterone and [6β-³H]-testosterone in HLM. When [1,2,6,7-³H]-testosterone was incubated with HLM in the presence of an NADPH regenerating system, [³H]-H₂O was formed in a time-dependent manner (FIG. 3A). Formation of tritiated water was dependent on NADPH. Product formation increased linearly with the concentration of microsomes up to a protein concentration of 0.5 mg/mL. Similar results were obtained using [6β-³H]-testosterone as substrate (FIG. 3B). The main differences between the two substrates were the signal to noise ratios and the fractional conversion rates. Signal to noise ratio is defined as the ratio between product counts obtained in the presence vs. absence of NADPH. The fractional conversion rate is expressed as percent of total radiolabelled substrate converted into tritiated water per unit time and per mg of microsomal protein. As summarized in Table 1, signal to noise ratios were about seven with [1,2,6,7-³H]-testosterone and about 20 with [6β-³H]-testosterone. Fractional conversion rates were about 0.6%/min/mg for [1,2,6,7-³H]-testosterone and 10- to 14-fold higher for [6β-³H]-testosterone.

TABLE 1
Signal to noise ratios (SN) and fractional conversion rates of tritiated
water formation from radiolabelled testosterone

| | Unlabelled testosterone (μM) | | | | | |
|---|---|---|---|---|---|---|
| | 10 | | | 60 | | |
| | | | Conversion rate | | | Conversion rate |
| Tracer | n | SN | (%/min/mg) | n | SN | (%/min/mg) |
| [1,2,6,7-³H]-testosterone | 4 | 6.2 ± 1.3 | 0.57 ± 0.23 | 8 | 7.3 ± 1.0 | 0.58 ± 0.06 |
| [6β-³H]-testosterone | 3 | 18 ± 9 | 5.9 ± 1.9 | 4 | 22 ± 7 | 9.8 ± 2.8 |

Data are mean values ± SEM;
n, number of experiments.

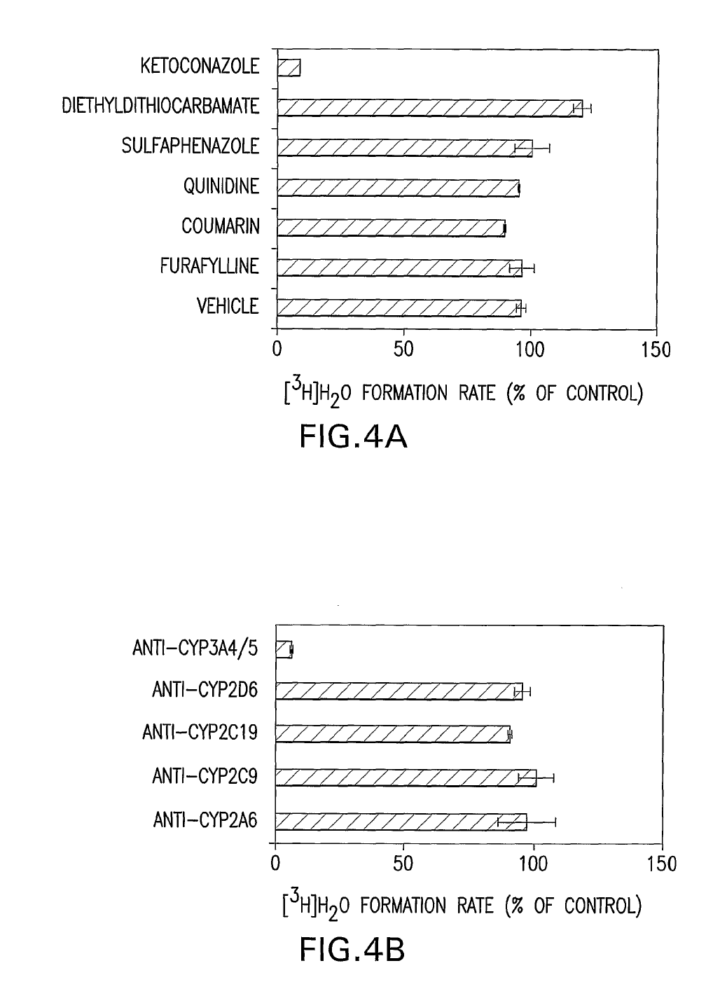

Effect of CYP inhibitors and anti-CYP antibodies. To determine which CYP isoform mediates product formation, reactions were performed in the presence or absence of a series of isoform-selective chemical inhibitors (Bourrie et al., 1996; Eagling et al., 1998) or monoclonal antibodies (Mei et al., 1999; Shou et al., 2000). Chemical inhibitors that were used were furafylline (CYP1A2 inhibitor), coumarin (CYP2A6 inhibitor), sulfaphenazole (CYP2C9 inhibitor), quinidine (CYP2D6 inhibitor), diethyldithiocarbamate (CYP2E1 inhibitor), and ketoconazole (CYP3A4/5 inhibitor). Monoclonal antibodies that were used were inhibitors of CYP2A6, CYP2C9, CYP2C19, CYP2D6, and CYP3A4/5. As shown in FIGS. 4A and 4B, none of these agents significantly affected formation of tritiated water from in HLM, with the exception of ketoconazole and the anti-CYP3A4/5 monoclonal antibody, which inhibited the reaction by over 90%. This showed that the assay was specific for detecting CYP3A4/5 activity.

Kinetic isotope effect. CYP3A4/5-mediated loss of tritium from [1,2,6,7-³H]-testosterone may occur as a consequence of hydroxylation at either the 6β- or 2β-positions(Waxman et al., ibid.; Yamazaki and Shimada, ibid. (1997)). The activity of testosterone 2β-hydroxylase in the batch of HLM used in the present experiments was about 11% of that of the 6β-hydroxylase (data not shown). The formation of both metabolites was mediated by CYP3A4/5, since it was completely inhibited by 10 μM ketoconazole over the entire substrate concentration range (data not shown). The relative proportions of label in the 6 [symbol] and 2 [symbol] positions of [1,2,6,7-³H]-testosterone are not known, and it cannot therefore be determined from which position the tritium loss occurred.

Following CYP3A4-mediated hydroxylation of [6β-³H]-testosterone, tritium can be lost only from the 6β position. To determine whether tritium substitution gives rise to a kinetic isotope effect, the rate of formation of [³H]-H₂O was compared to the rate of formation of 6β-hydroxytestosterone in the same reaction mixture. Considering that the proton lost upon hydroxylation of testosterone is formed stoichiometrically with 6,-hydroxytestosterone, the specific radioactivity of the tritiated water product can be calculated by dividing [³H]-H₂O product counts by the amount of 6β-hydroxytestosterone formed (Table 2). Together with the fractional conversion rate of tritiated tracer and its known initial specific radioactivity, this information can be used to calculate the kinetic isotope effect on V/K. As shown in Table 2, ^{T}V/K is 2.3, which is equal to the substrate/product specific radioactivity ratio.

TABLE 2
Tritium isotope effect for hydroxylation of [6β-³H]testosterone.

| Microsome concentration | | |
|---|---|---|
| (mg/mL) | SA₀/SA_{P} | T_{V/K} |
| 0.1 | 2.39 | 2.40 |
| 0.25 | 2.22 | 2.24 |
| 0.5 | 2.18 | 2.21 |
| 1.0 | 2.47 | 2.53 |
| | Average: 2.32 ± 0.14 | Average: 2.34 ± 0.15 |

Formation of [³H₂O] and 6β-hydroxytestosterone were determined at 9 different time points between 2 and 30 minutes, using 4 different concentrations of HLM. 6β-hydroxytestosterone was quantified by LC-MS/MS analysis. For each microsome concentration, data are mean values from all the time points.
SA₀, initial specific radioactivity of [6β-³H]testosterone;
SA_{P}, specific radioactivity of the product, as defined in the Experimental section above;
T_{V/K}, kinetic isotope effect.

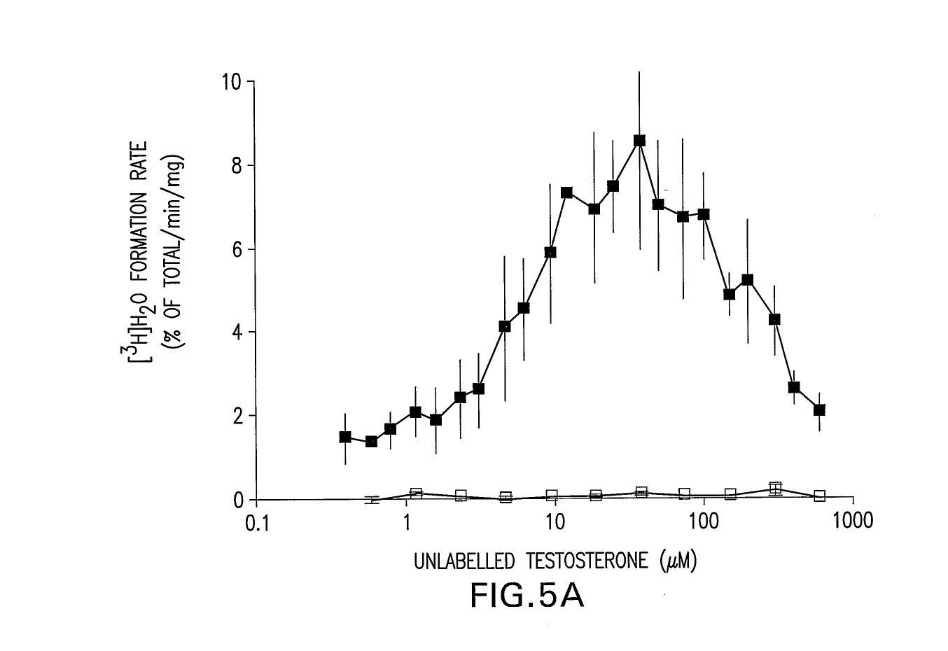

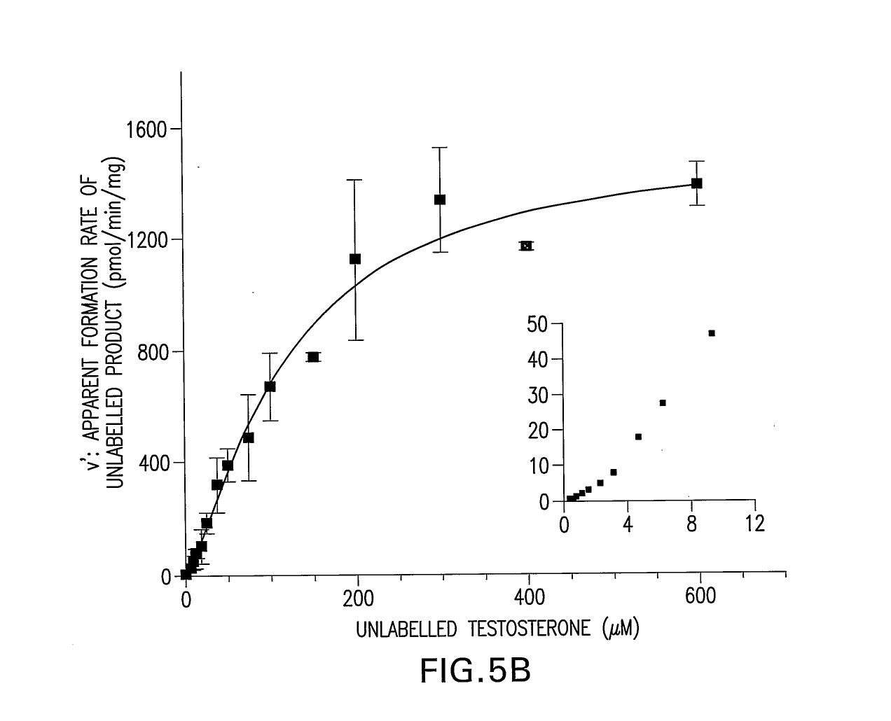

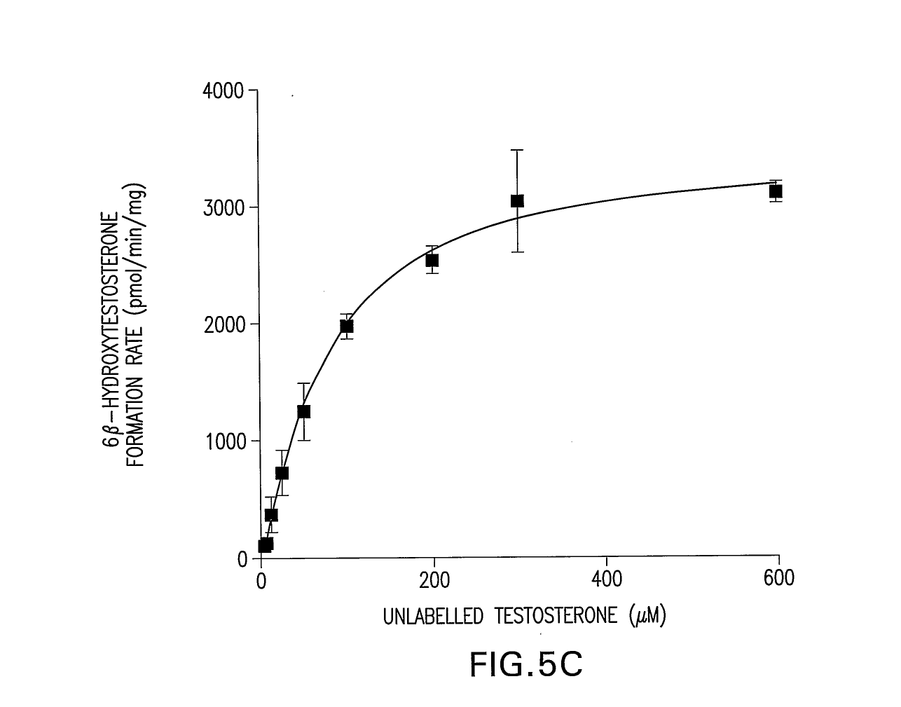

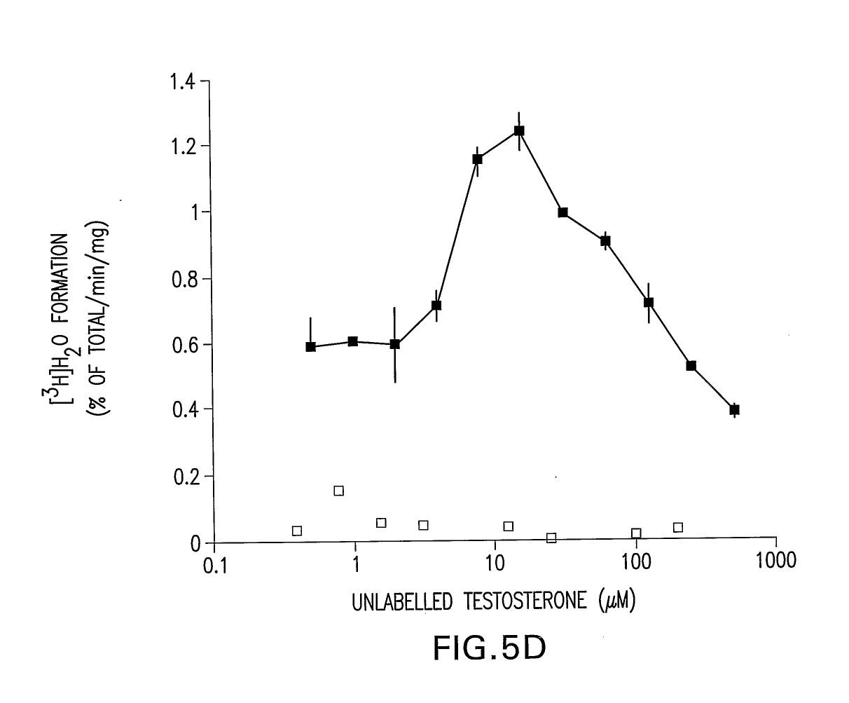

Competition between radiolabelled and unlabelled testosterone. The effect of unlabelled testosterone on the formation of tritiated water in HLM is depicted in FIG. 5A. The curve displays a “low dose hook”, i.e. product formation rate increased with increasing concentration of unlabelled substrate, reached a peak at a testosterone concentration of 30 to 40 μM, and then decreased. Since [6β-³H]-testosterone is used as an isotopic tracer, the formation rate of tritiated water (v*) is representative of that of unlabelled product, namely water derived from 6β-hydroxylation of testosterone (which is formed stoichiometrically with 6β-hydroxytestosterone). The dependence of v* on substrate concentration (S) can be used to obtain information about the dependence on substrate concentration of the unlabelled product, even if the latter is not measured directly. We define v′, the apparent formation rate of unlabelled product, as the formation rate of unlabelled product (v) divided by the kinetic isotope effect (See Experimental section above). As depicted in FIG. 5B, the curve of v′ vs S could be fitted to the Hill equation, with S₅₀=98 [symbol] μM, n=1.4±0.2, and V′max=1.3±0.1 nmol/min/mg (average ±SEM, n=3). The Hill coefficient was greater than 1, suggesting positive coöperativity. Indeed, at low substrate concentrations, a sigmoidal relationship was observed between v′ and S, as depicted in the inset of FIG. 5B.

The kinetics of 6β-hydroxytestosterone formation is depicted in FIG. 5C. The reaction had a S₅₀ of 65±17 μM, Vmax of 3.4±0.04 nmol/min/mg protein, and Hill coefficient of 1.5±0.2 (average ±SEM, n=5). Note that the ratio between V′ₘₐₓ and Vₘₐₓ is 2.6, which is close to the kinetic isotope effect on V/K, as expected from the definition of V′ₘₐₓ (See Experimental section above). The formation of tritiated water and of 6β-hydroxytestosterone was completely inhibited by ketoconazole, confirming that both products are formed via CYP3A4-mediated metabolism.

Similarly, substrate competition between a fixed concentration of [1,2,6,7-³H]-testosterone and increasing concentrations of unlabelled testosterone displayed a low-dose hook, and tritiated water formation from this substrate was completely inhibited by ketoconazole (FIG. 5D). Since neither the specific radioactivity of the tracer nor the proportion of product coming from the 2β- and 6β-positions are known, the formation rates of the corresponding unlabelled products cannot be determined from isotopic dilution studies.

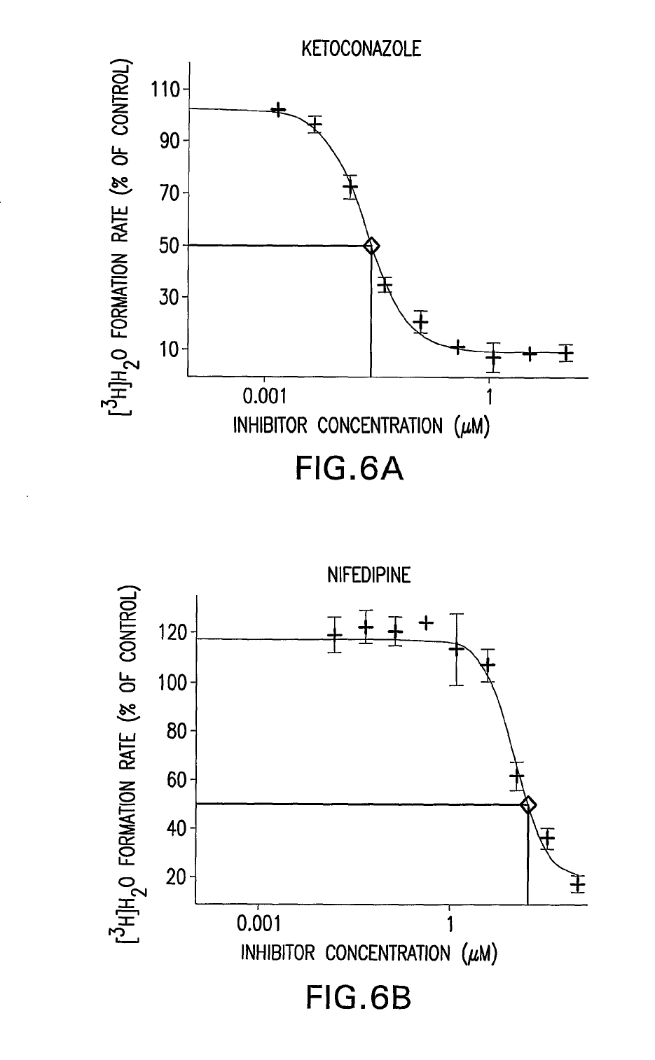

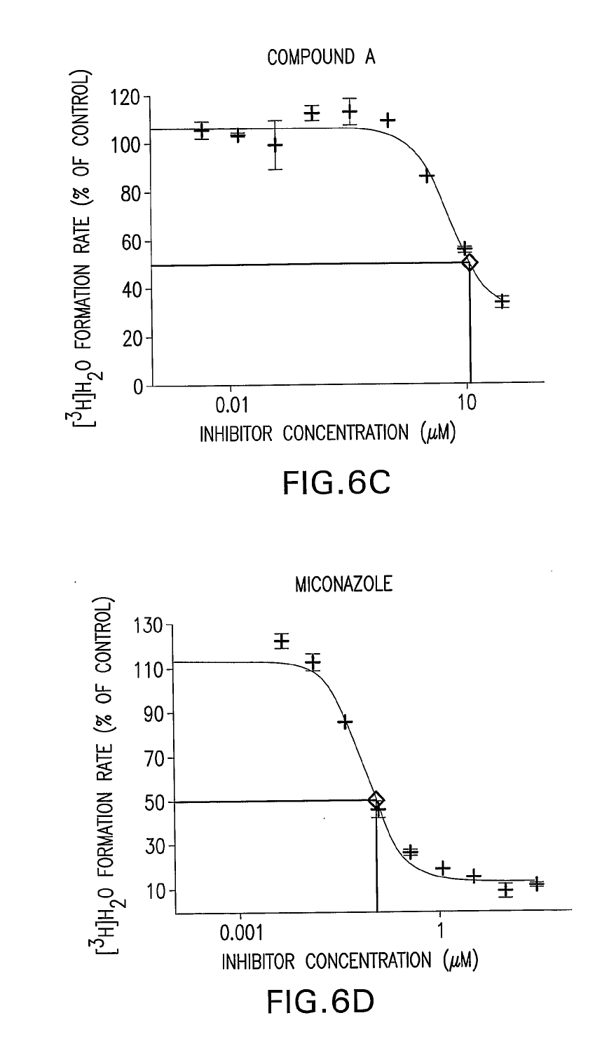

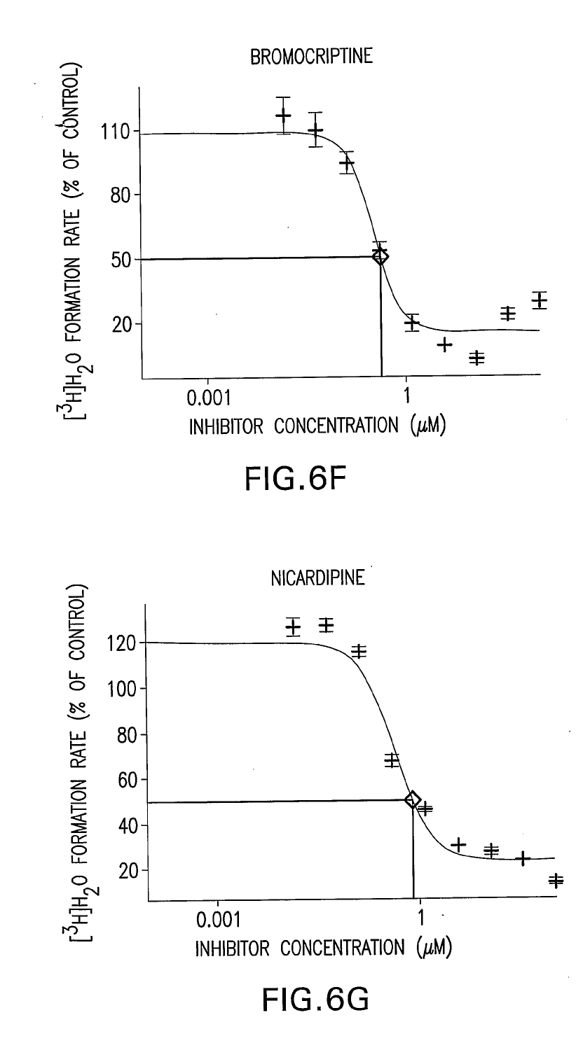

Kinetics of inhibition by CYP3A4/5 inhibitors. The effect of known inhibitors of CYP3A4/5 on the rate of formation of [³H]-H₂O from [1,2,6,7-³H]-testosterone and [6β-³H]-testosterone is shown in FIGS. 6A through 6F. IC₅₀ values are summarized in Table 3. With [1,2,6,7-³H]-testosterone, inhibition experiments were carried out in the presence of two different concentrations of unlabelled testosterone, 10 and 60 μM. No significant differences (less than 3.5-fold) in IC₅₀ values were observed at the two different substrate concentrations. IC₅₀ values determined with the two different tracers were very similar, with differences of less than two-fold. To confirm that IC₅₀ values obtained with the radiometric assay reflect those of the conventional assay, the effect of the inhibitors on [³H]—H₂O and 6β-hydroxytestosterone formation was determined in the same reaction mixture. As shown in Table 3, almost identical IC₅₀ values were obtained.

TABLE 3
IC₅₀ values of CYP3A4 inhibitors

| | IC₅₀ (μM) | | | |
|---|---|---|---|---|
| | | | | 6OHBT |
| | [³H]-H₂O formation | | | formation |
| | [1,2,6,7-³H]-testosterone | | [6β-³H]-testosterone | |
| Inhibitor | 10 μM Testo | 60 μM Testo | 60 μM Testo | 60 μM Testo |
| Ketoconazole | 0.026 ± 0.006 | 0.031 ± 0.001 | 0.01 ± 0.01 | 0.023 ± 0.003 |
| Miconazole | 0.12 ± 0.01 | 0.12 ± 0.01 | 0.06 ± 0.01 | 0.10 ± 0.01 |
| Bromocriptine | 0.43 ± 0.03 | 1.5 ± 0.2 | 1.2 ± 0.3 | 1.4 ± 0.01 |
| Nicardipine | 0.80 ± 0.03 | 0.29 ± 0.06 | 0.23 ± 0.04 | 0.21 ± 0.08 |
| Compound A | 11.5 ± 0.3 | 7.8 ± 0.3 | 8.3 ± 0.9 | 6.9 ± 0.9 |
| Nifedipine | 17.2 ± 4.7 | 12.7 ± 2.3 | 12.4 ± 3.1 | 11.5 ± 0.5 |

The effect of known inhibitors on the formation rate of [³H]-H₂O and 6β-hydroxytestosterone (6OHBT) was determined in the same reaction mixture, using as substrate either [1,2,6,7-³H]-testosterone or [6β-³H]-testosterone, in the presence of the indicated concentrations of unlabelled testosterone (Testo.). IC₅₀'s were calculated from full inhibition curves with at least 8concentration points.
Data shown are mean values ± SEM from 2-4 experiments.

CYP inhibitors tested in these experiments had to be dissolved in organic solvents. Since inhibitory effects of solvents on CYP3A4 activity have been reported (Chauret et al., Anal. Biochem. 276: 215-226 (1999); Hickman et al., Drug Metab. Dispos. 26: 207-215 (1998); Busby et al., Drug Metab. Dispos. 27: 246-249 (1999)), we assessed the effect of two different concentrations of DMSO on the release of [³H]-H₂O from [6β-³H]-testosterone and on IC₅₀ values of selected inhibitors. DMSO inhibited product formation, with about 70% and 50% of control activity remaining at solvent concentrations of 0.3% and 0.7% (v/v), respectively. However, DMSO had no effect on the IC₅₀ values of ethynylestradiol, ketoconazole, miconazole, bromocriptine, nicardipine, nifedipine, quinidine, and verapamil (data not shown). Inhibition assays can therefore be performed using DMSO concentrations between 0.3 and 0.7%, as long as care is taken to include solvent controls and activity data are expressed relative to these controls.

Comparison with conventional testosterone 6β hydroxylation assay for a large number of compounds. IC₅₀ values for 39 structurally diverse new chemical entities (NCEs) were determined in the radiometric assay using [6β-³H]-testosterone and compared to historic data that had been generated using the conventional testosterone 6β hydroxylation assay. The results of this comparison are depicted in Table 4.

TABLE 4
Comparison between IC₅₀ values in radiometric vs. conventional assays

| | |
|---|---|
| IC₅₀ (μM) | Ratio |

| Compound No. | conventional | radiometric | (conv./radio.) |
|---|---|---|---|
| 1 | <0.4 | 0.2 | na |
| 2 | 1.3 | 1.5 | 0.85 |
| 3 | 1.7 | 2.3 | 0.73 |
| 4 | 2.0 | 1.8 | 1.14 |
| 5 | 2.5 | 0.9 | 2.88 |
| 6 | 3.9 | 4.5 | 0.87 |
| 7 | 4.5 | 2.0 | 2.19 |
| 8 | 5.2 | 1.6 | 3.22 |
| 9 | 5.5 | 1.3 | 4.13 |
| 10 | 5.8 | 3.7 | 1.57 |
| 11 | 6.9 | 2.0 | 3.41 |
| 12 | 7.8 | 3.2 | 2.45 |
| 13 | 8.4 | 3.7 | 2.26 |
| 14 | 8.7 | 20.8 | 0.42 |
| 15 | 9.3 | 9.3 | 1.00 |
| 16 | 9.5 | 2.3 | 4.10 |
| 17 | 10.7 | 15.3 | 0.70 |
| 18 | 11.4 | 13.2 | 0.87 |
| 19 | 11.8 | 7.6 | 1.56 |
| 20 | 12.5 | 7.5 | 1.67 |
| 21 | 14.0 | 8.5 | 1.65 |
| 22 | 14.8 | 6.6 | 2.24 |
| 23 | 16.2 | 5.8 | 2.79 |
| 24 | 17.9 | 10.2 | 1.75 |
| 25 | 18.8 | 10.3 | 1.83 |
| 26 | 21.9 | 28.4 | 0.77 |
| 27 | 25.4 | 12.7 | 2.01 |
| 28 | 27.0 | 79.4 | 0.34 |
| 29 | 34.6 | 39.1 | 0.88 |
| 30 | 44.5 | 48.0 | 0.93 |
| 31 | 48.7 | >100. | na |
| 32 | >100 | >100. | na |
| 33 | >100 | >100. | na |
| 34 | >100 | >100. | na |
| 35 | >100 | 61.9 | na |
| 36 | >100 | 76.4 | na |
| 37 | >100 | 81.1 | na |
| 38 | >100 | 62.3 | na |
| 39 | >100 | 76.8 | na |

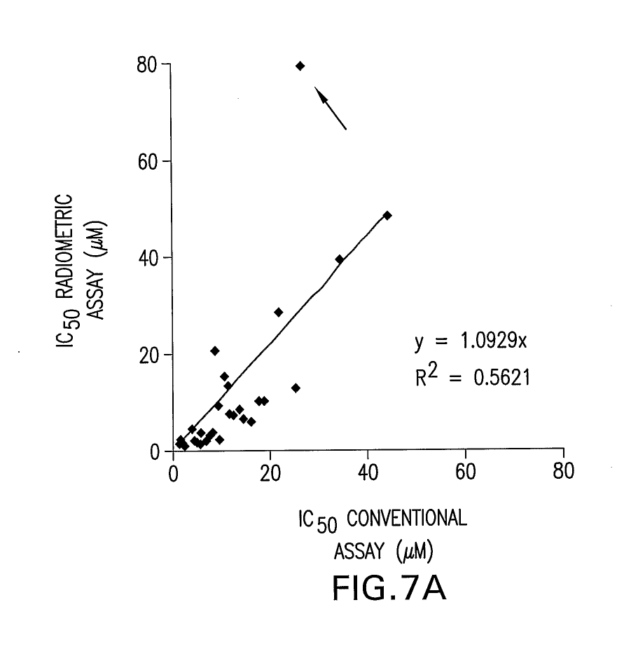

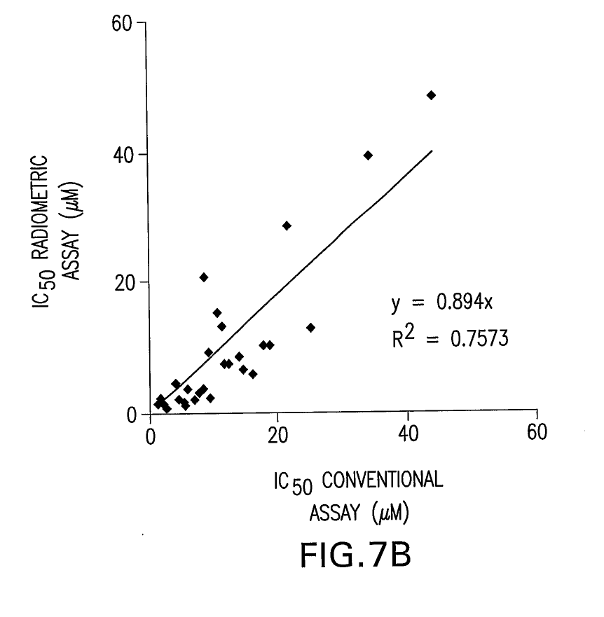

Three compounds had IC₅₀ values greater than 100 μM in both assays. Six compounds had IC₅₀ values greater than 100 μM in one assay and IC₅₀ values greater than 45 μM in the other assay. One compound had an IC₅₀ value less than 0.4 μM in one assay and 0.2 μM in the other assay. The IC₅₀ values of the remaining 29 compounds differed less than 4.2-fold between the two assays. Twenty five of these (86%) differed less than 3-fold. For the compounds with measurable IC₅₀ values in both assays, a plot of IC₅₀ values in the radiometric assay vs. IC₅₀ values in the conventional assay could be described by a straight line with a slope of 1.09 and a correlation coefficient r² of 0.562 (FIGS. 7A and 7B). The point that deviated most from the correlation line corresponded to a weak inhibitor (compound 28) with a less than 3-fold difference in IC₅₀s values between the two assays. When this compound was excluded from the analysis, the slope was 0.89 and the correlation coefficient increased to 0.757.

### Discussion

The release of tritium that accompanies hydroxylation of a substrate has been used to measure the activity of cytochrome P-450 enzymes (Thompson and Siiteri, J. Biol. Chem. 249: 5364-5372 (1974); Reed and Ohno, J. Biol. Chem. 251: 1625-1631 (1976); Miwa et al., J. Biol. Chem. 255: 6049-6054 (1980); Miyairi and Fishman, J. Biol. Chem. 260: 320-325 (1985); Osawa and Coon, Anal. Biochem. 164: 355-361 (1987)). Draper and co-workers described a CYP3A assay procedure based on this principle, in which tritiated water generated from CYPA-mediated metabolism of [1,2,6,7-³H]-testosterone was separated from the unreacted substrate by charcoal extraction (Draper et al., Drug Metab. Dispos. 26: 305-312 (1998)).

The present invention, which is amenable to automation, allows assays to be run in a multiwell format throughout the incubation and extraction steps and to collect the reaction product for scintillation counting in multiwell plates. The present invention differs from the above prior art assays in the following respects. First, the assay volume has been reduced from 500 μL to 200 μL and the microsome concentration reduced from 0.4 mg/mL to 0.25 mg/mL. Thus, the final amount of microsomes used is 50 μg instead of 200 μg. Second, in the preferred embodiment as demonstrated in herein, incubations are performed in a multiple well format, for example, a 96-well format. Third, tritiated water is separated from the tritiated testosterone substrate using multiple well solid phase extraction plates containing a support comprising a balanced ratio of hydrophilic and lipophilic polymers, such as the 96-well plates comprising the OASIS reverse phase resin. Finally, in preferred embodiments, the tritiated testosterone substrate contains tritium solely at the 6β position.

As shown in Example 1, the signal to noise ratios at testosterone concentrations of 10 to 60 μM (about 7-fold) are similar to those obtained by Draper et al. at a substrate concentration of 12.5 μM (6 to 7-fold in pooled ELM). However, reaction rates using the present embodiment of the assay are significantly higher, even when normalizing for the 3 to 6-fold higher amount of radiolabel used (250-500 nCi vs. 80 nCi in Draper et al.). Thus, Draper et al. reported a fractional conversion rate of [1,2,6,7-³]-testosterone into tritiated water of about 0.08%/min/mg protein using a concentration of 12.5 μM of unlabelled testosterone. In contrast, the conversion rate of [1,2,6,7-³H]-testosterone in the present assay is about 0.6%/min/mg at the same concentration of unlabelled testosterone (See above). The reason for this difference is not known; however, since the testosterone 6β hydroxylation activities were similar, the difference might be that different batches of commercially prepared [1,2,6,7-³H]-testosterone might contain different fractional amounts of tritium label in the 6β position.

Experiments with [1,2,6,7-³H]-testosterone have shown that the release of tritium-labeled water from [1,2,6,7-³H]-testosterone is catalyzed by recombinant CYP3A4 at a 6-fold higher rate than by recombinant CYP's 1A2, 2A6, 2B6, 2C9, 2C19, 2D6 and 2E1 (Draper et al., ibid). NADPH-dependent metabolism of unlabelled testosterone in HLM occurred mainly (greater than 75%) by 6β-hydroxylation and the rate of release of tritiated water from [1,2,6,7-³H]-testosterone correlated with that of testosterone 6β-hydroxylation in HLM from 12 different donor livers. On the basis of these findings, Draper et al. proposed that the NADPH-dependent release of tritium from [1,2,6,7-³H]-testosterone in HLM could be used as a probe reaction for CYP3A4 activity (Draper et al., ibid). Convincing validation of this claim would come from a demonstration that the reaction can be completely inhibited by specific CYP3A4 inhibitors, but not by inhibitors of other isoforms. The only inhibitor used in the experiments of Draper et al. was the competitive substrate erythromycin, which displayed a weak IC₅₀ of 130 μM. Other authors have reported that erythromycin and testosterone only weakly inhibit the metabolism of each other (Wang et al., Drug Metab. Dispos. 25: 502-507 (1997)). Therefore, the results with erythromycin do not adequately demonstrate CYP3A4 specificity of the assay. To demonstrate that the assay is specific for CYP3A4, monoclonal antibodies that specifically neutralized the activity of CYP3A4/5 or other isoforms (Mei et al., J. Pharmacol. Exp. Ther. 291:749-759 (1999); Shou et al., Curr. Drug Metab. 2: 17-36 (2000)) (CYPs 2A6, 2C9, 2C19 and 2D6), as well as specific chemical inhibitors of CYP's 3A4/5, 1A2, 2A6, 2C9, 2D6, and 2E1 were used. As shown in herein, among all these agents, only ketoconazole and an anti-CYP3A4/5 monoclonal antibody potently inhibited release of tritiated water from [1,2,6,7-³H]-testosterone in HLM, thereby demonstrating that the hydroxylation reaction is mediated almost exclusively (greater than 95%) by CYP3A4/5.

Further proof came from the results shown in herein of a more detailed analysis of the inhibitory potencies of a series of CYP3A4/5 inhibitors. Ketoconazole, nifedipine, bromocriptine, miconazole, nicardipine and compound A, a CYP3A4 inhibitor synthesized at MRL, inhibited release of tritiated water from [1,2,6,7-³H]-testosterone with IC₅₀ values that were not significantly different from those for inhibition of testosterone 6β hydroxylation, measured by LC-MS/MS in the same reaction mixture. Taken together, these results demonstrate that the assay disclosed herein faithfully measures the activity of microsomal CYP3A4/5 mediated hydroxylation of testosterone and that it can be used to analyze the inhibitory potencies of investigational drugs.

Draper and co-workers previously reported that water release from [1,2,6,7-³H]-testosterone and 6β-hydroxylation exhibited similar Kₘ and Vₘₐₓ values based on the assumption that the reaction of the tritiated tracer proceeds without any isotope effect and that formation of one equivalent of (unlabelled) 6β-hydroxytestosterone corresponds to formation of one equivalent of tritiated water. However, as discussed below, the assumption that hydroxylation of the tritiated tracer proceeds in the absence of a kinetic isotope effect was not valid, rendering it not possible to determine molar reaction velocities using commercially available [1,2,6,7-³H]-testosterone. In order to calculate the velocity of product formation from the tritiated substrate, it is necessary to know the specific activity of radiolabel present in the 6 [symbol] position. For commercial [1,2,6,7-³H]-testosterone, this information is not available. According to the information supplied by the vendor, about 20% of the label is in the 1α position, 19% in 2α, 20% in 6α, 25% in 7β, 10% in (1β plus 7β) and less than 6% in positions 2β and 6β, but the exact proportion of label present in the 6β position is not known.

Aside from the inability to determine kinetic reaction parameters using commercially available [1,2,6,7-³H]-testosterone, the use of the commercially available [1,2,6,7-³H]-testosterone as a CYP3A probe presents several additional drawbacks. Because of the very low amount of label in the 6β position (less than 6%), it was necessary to use quite high amounts of radioactive substrate (0.5−1×10⁶ dpm/assay) to obtain adequate product counts. This has a significant impact on the cost of the assay and renders it difficult to miniaturize the product counting procedure, for example, to use 96-well scintillation counters. Furthermore, it cannot be formally excluded that tritium loss from positions other than the 6 [symbol] position contributes to product formation. The availability of testosterone specifically labeled in the 6 [symbol] position represents a significant advantage with regard to these issues. When using this novel tracer, the assay volume can be further reduced to 100 μL and the amount of microsomes to 25 μg, and reasonable product counts (2000 to 4000 dpm) could be obtained with as little as 100,000 dpm/assay of substrate probe. The signal to noise ratio was significantly improved to about 20-fold. Formation of tritiated water from [6β-³H]-testosterone was NADPH-dependent and inhibited by a series of CYP3A/5 inhibitors with IC₅₀ values that were not significantly different from those obtained with [1,2,6,7-³H]-testosterone. The improved assay of the present invention thus represents a true high-throughput radiometric version of the classical testosterone 6β-hydroxylation assay and is suitable for rapid screening of the inhibitory potential of investigational drugs, as further discussed below.

Using [6β-³H]-testosterone, whose specific radioactivity is known, as substrate probe allows determination of a kinetic isotope effect, ^{T}V/K (See above). The large disparity between the concentrations of tritiated and unlabelled substrate makes it not possible to measure their separate reaction velocities. When trace concentrations of a tritiated substrate are used to measure reaction velocities in the presence of competing unlabelled substrate, it is possible to measure the kinetic isotope effect on the V/K ratio (Northrop, Meth. Enzymol. 87:607-625 (1982)). By measuring formation of tritiated water and 6β-hydroxytestosterone in the same reaction mixture, the specific activity of the tritiated water product can be calculated and compared to the initial specific radioactivity of the substrate, and these values can be used to derive ^{T}V/K. The kinetic isotope effect on ^{T}V/K was 2.3-fold. It was previously reported that 6β-hydroxylation of deuterated testosterone proceeds in the absence of a kinetic isotope effect (Bjorkhem, Eur. J. Biochem. 27: 354-363 (1972)). This is most likely due to the difference between C-D and C-T bond energies. The kinetic isotope effect reduces the reaction velocity for the tritiated probe but does not affect the inhibitory potencies of competitive CYP3A inhibitors, as demonstrated by the observation that IC₅₀ values for inhibition of tritiated water formation were not significantly different from those for 60-hydroxytestosterone formation and were not modified by 6-fold isotopic dilution of the substrate probe.

When reaction velocity is measured in the presence of a fixed amount of radiolabelled substrate and increasing concentrations of unlabelled substrate, product counts are expected to decrease when the concentration of unlabelled substrate becomes sufficiently high to compete with binding of the tracer to the active site of the enzyme. As shown in FIG. 5A through 5B, at low concentrations of unlabelled testosterone, the formation of tritiated water was actually enhanced as unlabelled substrate increases and starts to decrease only at high concentrations (greater than 40 μM) of unlabelled substrate. This effect, called “low dose hook”, is characteristic for positively coöperative ligand displacement interactions (De Lean and Rodbard, Recept.: Compr. Treatise 1: 143-192 (1979)). The reason for the increased formation of tritiated product is that at low substrate concentrations, the reaction velocity of a positively coöperative enzyme increases more than dose-proportionally with increasing substrate concentration. When the apparent formation rate of unlabelled product was calculated from the tracer competition data and plotted against the substrate concentration, a sigmoidal relationship was observed, confirming that the reaction displays positive coöperativity. Data could be fitted to the Hill equation, with a Hill coefficient of 1.34 and a S₅₀ of 120 μM. Measurement of 6β-hydroxytestosterone formation under the same conditions demonstrates that reaction kinetics of unlabelled testosterone indeed display positive coöperativity, with an apparent Hill coefficient of 1.35, and an S₅₀ of 101 μM. A Hill coefficient of 1.3 has previously been reported for CYP3A4-medited 6β-hydroxylation of testosterone (Ueng et al., Biochemistry 36: 370-381 (1997)). The good agreement between kinetic parameters obtained from the tracer competition data with those determined by direct measurement of unlabelled product reinforces the notion that formation of tritiated water from [6β-³H]-testosterone in HLM is mediated by the same mechanism as formation of 6β-hydroxytestosterone. A low dose hook was also observed using [1,2,6,7-³H]-testosterone as substrate. It should be noted that hyperbolic (non-sigmoidal) kinetics have been reported for this tracer (Draper et al. ibid). The most likely explanation for this apparent discrepancy is that the “low dose hook” was observed in the present studies only at substrate concentrations between 0.4 and 20 μM while Draper studied reaction kinetics at substrate concentrations greater than 14 μM.

To validate the use of the new assay as a screening method for CYP3A4/5 inhibition, IC₅₀ values were determined for a large number of structurally diverse investigational compounds and compared the results with those obtained in the classical testosterone 6β-hydroxylation assay (with product quantification by LC-MS/MS). The results of this analysis indicate that IC₅₀ values obtained with the assay of the present invention are very similar to those of the conventional assay. IC₅₀ values differed less than 4-fold, and for 86% of the compounds less than 3-fold, which is quite remarkable considering that historical data for the 6β hydroxylation assay were used for this comparison. Most importantly, not a single compound out of the 39 tested would have been misclassified as either a strong or weak inhibitor based on the results of the radiometric assay.

A detailed comparison for a large number of compounds of IC₅₀ values obtained with a fluorogenic CYP3A4/5 probe vs. testosterone revealed that the correlation between these assays was relatively poor. The authors of this study recommended that screening with fluorogenic probes should be followed up by studies with conventional substrates (Cohen et al., Drug Metab. Dispos. 31: 1005-1015 (2003)). Therefore, the assay of the present invention, which combines the advantages of speed, high throughput, and the use of a conventional substrate, circumvents this issue and is a valuable tool for rapidly determining the potential of compounds to inhibit CYP3A4 in a drug discovery setting.

### EXAMPLE 2

This example illustrates the use of the present invention to determine and quantify the enzymatic activity and the effect of CYP3A4/5 inhibitors in intact hepatocytes.

Human hepatocytes were prepared from fresh liver samples (surgical waste obtained from a local hospital). Hepatocytes were isolated and cryopreserved in liquid nitrogen according to established protocols (See for example, Hengstler et al., Drug Metab. Rev. 32: 81-118 (2000); Ferrini et al., Methods Mol. Biol. 107: 341-52 (1998)). Cells were thawed and incubated for one hour at 37° C. in a shaking water bath under a humidified atmosphere of 5% CO₂, 95% oxygen, in 12-well culture plates. Each culture well contained one million cells, 1 mL of hepatocyte culture medium (HCM) (Dich and Grunnet, in Methods in Molecular Biology, Vol. 5: Animal Cell Culture (Pollard and Walker, eds) pp. 161-176, Humana Press, Clifton, N.J. (1989)), 200 μM unlabelled testosterone, and 370,000 dpm of [6β-³H]-testosterone. When assaying an analyte for inhibition of CYP3A4/5 activity, the analyte is added to the above reaction. In this example, the inhibitor 10 μM ketoconazole was added to parallel incubations. Control incubations are also performed comprising a known inhibitor such as 10 μM ketoconazole or the vehicle for the analyte. After one hour, aliquots of the incubation medium were loaded onto individual wells of preconditioned 30 mg OASIS plates, which were washed two times with 200 μL of water. For each well, the flow-through was combined with the water washes and counted in a beta-counter after addition of scintillation fluid.

To measure the rate of formation of 6β-hydroxytestosterone, 6β-hydroxytestosterone was eluted from the OASIS plates with 1 mL of methanol, dried under N₂, and reconstituted in 200 μL of 50% acetonitrile/water (50:50) containing 0.1% of formic acid. Aliquots were injected into an HPLC-MS/MS system for quantification of 6β-hydroxytestosterone. Quantification was based on comparison of peak areas with those of a standard curve that was treated and extracted exactly like unknown samples. The apparent formation rate of [³H]-H₂O was calculated by dividing product counts by the specific radioactivity of testosterone, as described in Example 1. Results are shown in Table 5.

TABLE 5

| | Apparent | Formation rate of 6β- |
|---|---|---|
| | formation rate of [³H]-H₂O | hydroxytestosterone |
| Cell treatment | (nmol/hr/million cells) | (nmol/hr/million cells) |
| None | 7.0 | 10.2 |
| Ketoconazole | 0.0 | Not detected |

The activity determined using the radiometric assay of the present invention was slightly (30%) lower than that determined with the LC-MS/MS assay, probably due to a moderate kinetic tritium isotope effect as described in Example 1. Formation of [³H]-H₂O from [6β-³H]-testosterone was completely inhibited in the presence of ketoconazole, demonstrating that the assay can also be used to determine the effect of CYP3A4/5 inhibitors in intact human hepatocytes.

### EXAMPLE 3

This example illustrates the use of the present invention to determine and quantify the effect of CYP3A4/5 inducers in hepatocytes.

Cryopreserved human hepatocytes from two different donors were obtained from Tissue Transformation Technologies (Edison, N.J.). Cells (ca. 320,000) were plated in 24-well collagen-coated culture plates and maintained at 37° C. in a humidified atmosphere of 5% CO₂, 95% air, in hepatocyte culture medium (HCM) (Dich and Grunnet, ibid.) supplemented with ITS+(Collaborative Research, Waltham, Mass.). Twenty-four hours later, the culture medium for each well of cells was removed, fresh HCM with ITS was added, and cells were treated with either vehicle (control), rifampicin (positive control), or analyte being tested for ability to induce CYP3A4/5 activity for 48 hours. CYP3A4/5 enzyme activity was then determined as follows.

For each well, the medium was removed and the cells were incubated in 0.5 mL of Hank's balanced salt solution (HBSS) containing 10 mM Hepes, pH 7.4, 60 μM unlabelled testosterone, and ca. 200,000 dpm of [6β-³H]-testosterone for 1 hour at 37° C. For each, parallel incubations were also performed in the presence of 10 V ketoconazole to ascertain that enzyme activity was specifically mediated by CYP3A4/5. The incubation medium was then loaded onto individual wells of preconditioned 30 mg OASIS plates, which were washed two times with 200 μL of water. For each well, the flow-through was combined with the water washes and counted in a beta-counter after addition of scintillation fluid. 6-p-hydroxytestosterone was eluted from the OASIS plates with 1 mL of methanol, dried under N₂, and reconstituted in 200 uL of 50% acetonitrile/water (50:50) containing 0.1% of formic acid. Aliquots were injected into an HPLC-MS/MS system for quantification of 6β-hydroxytestosterone. Quantification was based on comparison of peak areas with those of a standard curve that was treated and extracted exactly like unknown samples.

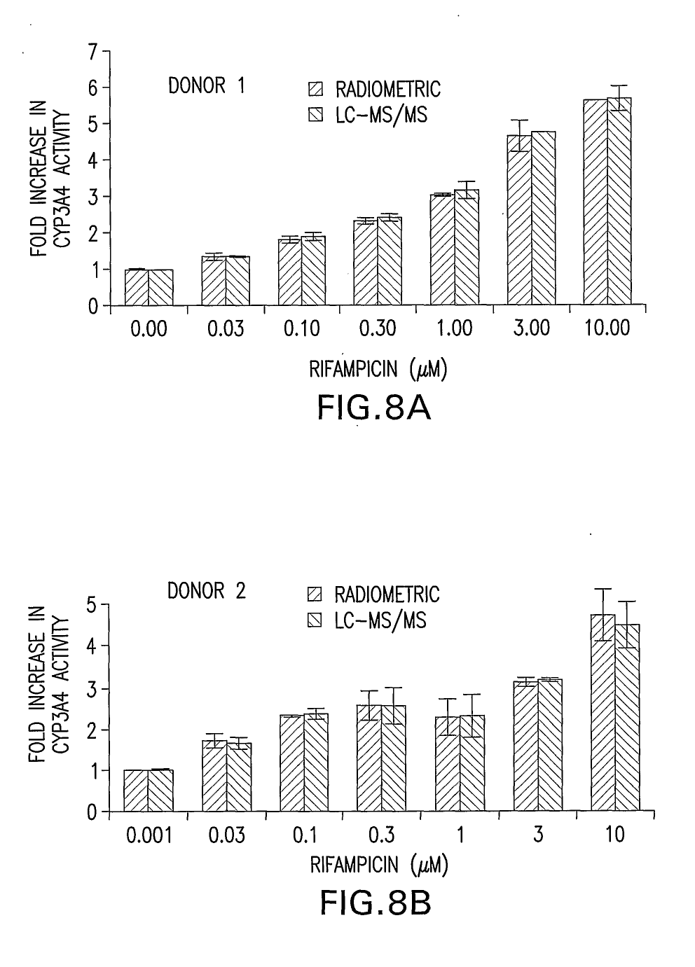

As shown in FIGS. 8A and 8B (results for donors 1 and 2, respectively), treatment of cultured human hepatocytes for 48 hours with rifampicin caused a dose-dependent increase in CYP3A4/5 enzymatic activity. CYP3A4/5 activity was determined by measuring [³H]-H₂O formation from [6β-³H]-testosterone and, in parallel, formation of 6β-hydroxytestosterone in the same cell incubations. Results are expressed as percent of metabolite formation relative to control (cells not treated with rifampicin). The effect of rifampicin measured by the radiometric method of the present invention was not significantly different from that measured by the conventional (LC-MS/MS) assay. Formation of both metabolites was almost completely inhibited when incubations were performed in the presence of 10 μM ketoconazole (data not shown), confirming CYP3A4/5 specificity of the reaction. These results show that the assay of the present invention can be used to determine the effect of CYP3A4 inducers in intact cells, and in particular, in cultured human hepatocytes.

### EXAMPLE 4

This example shows a time-dependent CYP3A4/5 assay. All incubations were performed in a 96 well box plus polypropylene tubes (Greiner-International PBI).

The preincubation step was performed as follows. Preincubation mixtures contained: 30 μL HLM (3.3 mg/ml of protein, preferred final concentration 2 mg/mL; range 0.1 to 5 mg/mL), 1 μL of test analyte (dissolved in 35% DMSO, 65% Methanol), 9 μL of assay buffer (0.1 M potassium phosphate, pH 7.6). Preincubations were started by adding 10 μL of NADPH regenerating system (5 mM NADPH, 25 mM Glucose-6-phosphate, 17 mM MgCl₂, 5 U/mL Glucose-6-phosphate dehydrogenase, in assay buffer). Preincubations were started at different times in reverse order (longest preincubation was started first, shortest preincubation was started last). Mixtures were preincubated in a shaking water bath for 0-30 minutes at 37° C.

Determination of remaining activity was as follows. The second incubation was started by 10-fold dilution of the preincubation mixtures with 450 μL of assay buffer containing [60-3H]testosterone (about 800,000 dpm), 200H unlabelled testosterone and 1 mM NADPH. Incubations were performed in a shaking water bath for 10 min at 37° C. Reactions were stopped by addition of 50 μL of 1N HCl. Plates were centrifuged at room temperature at 2800 rpm for 15 minutes. Three hundred μL of supernatant were loaded on a preconditioned 30 mg OASIS plate. The flow-through was collected and aliquots of 120 μL were transferred into 96 well scintillation counting plates (Packard). 180 μL of MICROSCINT 40 scintillation fluid was added and plates were sealed, shaken, and counted in a Packard TOPCOUNT scintillation counter.

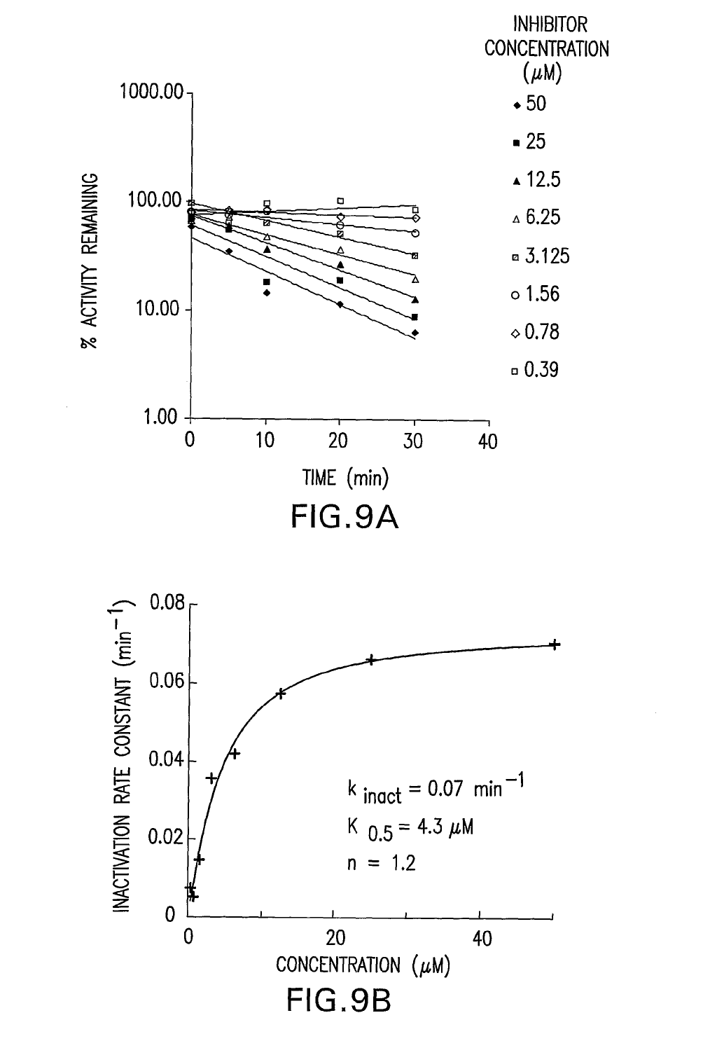

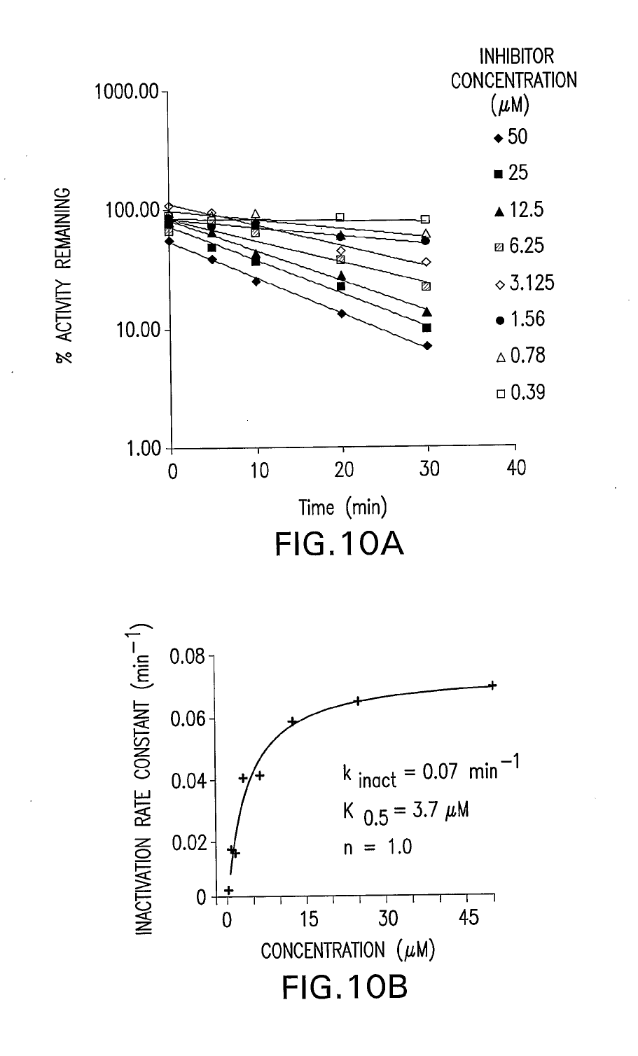

FIGS. 9A and 9B show the results of a typical experiment using radiometric detection of tritiated water to quantify CYP3A4/5-mediated testosterone 6β-hydroxylation. The time-dependent inhibitor used was mifepristone. FIGS. 10A and 10B show that similar results were obtained when testosterone 6β-hydroxylase activity was determined by LC-MS/MS analysis. Table 6 summarizes the kinetic parameters obtained for different time-dependent CYP3A4/5 inhibitors. For all compounds tested, results obtained by radiometric assay were similar to those obtained by LC-MS/MS assay, demonstrating that the radiometric assay can be used to assess the potential of compounds to act as time-dependent CYP3A4/5 inhibitors.

TABLE 6
Kinetic parameters for time-dependent CYP3A4/5 inhibitors obtained
using radiometric vs. conventional
(LC-MS/MS) testosterone 6β-hydroxylation assay.

| | k_{inact} (min⁻¹) | | K_{0.5} (μM) | | n | |
|---|---|---|---|---|---|---|
| Compound | Radiometric | LC-MS | Radiometric | LC-MS | Radiometric | LC-MS |
| Verapamil | 0.07 | 0.07 | 6.7 | 5.7 | 1 | 1 |
| Nicardipine | 0.38 | 0.17 | 2.7 | 3.0 | 2.5 | 2.7 |
| Troleandomycin | 0.2 | 0.3 | 0.5 | 0.8 | 0.7 | 1.3 |
| Mibefradil | 0.64 | 0.67 | 4.9 | 6.4 | 1 | 1 |
| Ritonavir | 0.19 | 0.19 | 1.2 | 1.4 | 2.1 | 2.1 |
| Mifepristone | 0.07 | 0.07 | 4.3 | 3.7 | 1.2 | 1 |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for identifing an analyte that inhibits activity of cytochrome 3A4 or 3A5 (CYP3A4/5), which comprises:
(a) providing a mixture comprising CYP3A4/5, tritium-labeled testosterone labeled with tritium at the 6β position, NADPH, and the analyte;
(b) incubating the mixture for a time sufficient for the CYP3A4/5 to hydroxylate the tritium-labeled testosterone at the 6β position;
(c) removing the CYP3A4/5 from the mixture;
(d) applying the mixture to a sorbent which preferentially binds non-polar compounds to remove the tritium-labeled testosterone from the mixture; and
(e) measuring amount of the tritium in the mixture of step (d) with the tritium-labeled testosterone removed, wherein a decrease in the amount of the tritium indicates that the analyte inhibits activity of the CYP3A4/5.

2. The method of claim 1 wherein the sorbent is selected from the group consisting of water-wettable polymers formed by copolymerizing at least one hydrophilic monomer and at least one lipophilic monomer in a ratio sufficient for the polymer to be water-wettable and effective at retaining organic solutes thereon and silica substrates comprising a non-polar group bonded to the silica substrate.

3. The method of claim 2 wherein the sorbent is poly(vinylbenzene-co-N-vinylpyrrolidone.

4. The method of claim 1 wherein the tritium-labeled testosterone is labeled at the 1, 2, 6β, and 7 positions.

5. The method of claim 1 wherein the tritium-labeled testosterone is labeled solely at the 6β position.

6. The method of claim 1 wherein the aqueous mixture further comprises an NADPH regenerating system.

7. The method of claim 1 wherein the tritium in the mixture in step (e) is compared to the amount of tritium in the mixture from a control mixture comprising CYP3A4/5, testosterone labeled with tritium in the 6β position, and NADPH, and not the analyte.

8. The method of claim 1 wherein the CYP3A4/5 is provided in microsomes.

9. The method of claim 7 wherein the microsomes are human liver microsomes.

10. The method of claim 7 wherein the microsomes are produced from cells selected from the group consisting of mammalian and insect cells, wherein the cells include a vector expressing the CYP3A4/5.

11-12. (canceled)

13. A method for determining the activity of cytochrome 3A4 or 3A5 (CYP3A4/5) in hepatocytes, which comprises:
(a) providing a culture of the hepatocytes;
(b) incubating the hepatocytes in a medium comprising testosterone labeled with tritium at the 6β position for a time sufficient for the CYP3A4/5 to hydroxylate the tritium-labeled testosterone at the 6β position;
(c) removing the medium from the culture of hepatocytes;
(d) applying the medium to a sorbent which preferentially binds non-polar compounds to remove the tritium-labeled testosterone from the medium; and
(e) measuring amount of the tritium in the medium of step (d) with the tritium-labeled testosterone removed, which determines the activity of the CYP3A4/5 in the hepatocytes.

14. The method of claim 13 wherein the sorbent is selected from the group consisting of water-wettable polymers formed by copolymerizing at least one hydrophilic monomer and at least one lipophilic monomer in a ratio sufficient for the polymer to be water-wettable and effective at retaining organic solutes thereon, and silica substrates comprising a non-polar group bonded to the silica substrate.

15-18. (canceled)

19. A method for identifying an analyte that irreversibly inhibits activity of cytochrome 3A4 or 3A5 (CYP3A4/5), which comprises:
(a) providing a mixture comprising CYP3A4/5, NADPH regenerating system, and the analyte;
(b) incubating the mixture for different times;
(c) diluting the mixture and then adding to the diluted mixture tritium-labeled testosterone labeled with tritium at the 6β position and NADPH;
(d) incubating the diluted mixture for a time sufficient for the CYP3A4/5 to hydroxylate the tritium-labeled testosterone at the 6, position;
(e) removing the CYP3A4/5 from the mixture;
(f) applying the mixture to a sorbent which preferentially binds non-polar compounds to remove the tritium-labeled testosterone from the mixture; and
(g) measuring amount of the tritium in the mixture of step (d) with the tritium-labeled testosterone removed, wherein a decrease in the amount of the tritium indicates that the analyte irreversibly inhibits activity of the CYP3A4/5.

20. The method of claim 19 wherein the sorbent is selected from the group consisting of water-wettable polymers formed by copolymerizing at least one hydrophilic monomer and at least one lipophilic monomer in a ratio sufficient for the polymer to be water-wettable and effective at retaining organic solutes thereon, and silica substrates comprising a non-polar group bonded to the silica substrate.
